SEQUENCE LISTING (2 sequences)

SEQ ID NO: 1 - DNA, 46 bp, Artificial Sequence
  Synthetic nucleic acid sequence
ccttggcacccgagaattccatccgtcttgcgccggtccaagaatt

SEQ ID NO: 2 - DNA, 55 bp, Artificial Sequence
  Synthetic nucleic acid sequence
tcacctctagcggcgcaatacgaataaaaaaaaaaaaaaaaaaaaaaaaaaaaaa

(19) United States
(12) Patent Application Publication
(10) Pub. No.: US 2024/0218432 A1
(43) Pub. Date: Jul. 4, 2024

(54) METHODS FOR ASSESSING SAMPLE QUALITY PRIOR TO SPATIAL ANALYSIS USING TEMPLATED LIGATION

(71) Applicant: 10x Genomics, Inc., Pleasonton, CA (US)

(72) Inventor: Paulius Mielinis, Stockholm (SE)

(21) Appl. No.: 18/287,560

(22) PCT Filed: Apr. 20, 2022
(86) PCT No.: PCT/US2022/025531

§ 371 Date: Oct. 19, 2023

Related U.S. Application Data
(60) Provisional application No. 63/177,179, filed on Apr. 20, 2021.

(51) Int. Cl. C12Q 1/6841

(52) U.S. Cl. CPC C12Q 1/6841

(57) ABSTRACT

Provided herein are methods of testing a biological sample for efficacy of detection of a target nucleic acid where the method includes RNA templated ligation including generating a sequence that is complementary to the hybridized ligation product that includes labelled nucleotides and detecting a signal corresponding to the ligation product on the substrate, thereby determining the efficacy of detection of the target nucleic acid.

### CROSS-REFERENCE TO RELATED APPLICATIONS

This application is an International Application, which claims the benefit to U.S. Provisional Patent Application No. 63/177,179, filed Apr. 20, 2021. The contents of this priority application are incorporated herein by reference in their entireties.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes (e.g., target nucleic acids) in the context of intact tissue or a portion of a tissue (e.g., tissue section), or provide significant analyte data from individual, single cells, but fails to provide information regarding the position of the single cells from the originating biological sample (e.g., tissue).

Generally, targeting a particular target nucleic acid in a biological sample utilizes a capture probe that targets a common transcript sequence such as a poly(A) mRNA-like tail. However, this approach is capable of detecting a high number of off target nucleic acids. Methods such as RNA-templated ligation offer an alternative to indiscriminant capture of a common transcript sequence. See. e.g., Yeakley, PLOS One, 25:12(5): e0178302 (2017), which is incorporated by reference in its entirety. However, there remains a need to develop an alternative to common transcript sequence (e.g., poly(A) mRNA-like tail) capture of target nucleic acids that is capable of detecting a target nucleic acid(s) in an entire transcriptome while providing information regarding the spatial location and abundance of a target nucleic acid. Typically, this requires sequencing which can be time and resource intensive. Therefore, there is a need to assess sample (biological sample) quality and assay sensitivity prior to spatial analysis using RNA-templated ligation.

### SUMMARY

Targeted RNA capture is an attractive alternative to poly(A) mRNA capture in order to interrogate spatial gene expression in a sample (e.g., an FFPE tissue). Compared to poly(A) mRNA capture, targeted RNA capture as described herein is less affected by RNA degradation associated with FFPE fixation compared to methods dependent on oligo-dT capture and reverse transcription of mRNA. Further targeted RNA capture as described herein allows for sensitive measurement of specific genes of interest that otherwise might be missed with a whole transcriptomic approach. Targeted RNA capture can be used to capture a defined set of RNA molecules of interest, or it can be used at a whole transcriptome level, or anything in between. When combined with the spatial methods disclosed herein, the location and abundance of the RNA targets can be determined.

Assessing quality of a biological sample and determining which conditions effectively allow for detection of a target RNA can identify biological samples conducive to targeted RNA capture. As disclosed herein, one can vary conditions such as altering permeabilization conditions and adjusting buffer and enzyme concentrations that will enable greater capture and/or decreased degradation of RNA in the biological sample. Permeabilization of tissues is one important aspect of performing spatial transcriptomics. If a tissue is not efficiently or completely permeabilized such that the probes used for targeted RNA capture are impeded from hybridizing to their target sequences in the biological sample, then complete ligation products may not be generated and therefore not captured by capture probes. As such, target spatial information may be weak with low resolution as a result. Additionally, as tissues differ, so might their permeabilization conditions. As such, optimizing the best permeabilization condition for any particular tissue (e.g., on a test section of a tissue) may be needed for complete ligation product capture. The methods described herein enable assessment of the biological sample following optimization of permeabilization conditions that will allow for ligation product capture.

In one aspect, this disclosure features methods of testing efficacy of detection of a target nucleic acid in a biological sample, the method comprising: (a) contacting the biological sample on an array comprising a plurality of capture probes, wherein a capture probe of the plurality comprises a capture domain; (b) contacting a first probe and a second probe with the biological sample, wherein the first probe and the second probe each comprise sequences that are substantially complementary to sequences of the target nucleic acid, and wherein the second probe comprises a capture probe capture domain; (c) hybridizing the first probe and the second probe to the target nucleic acid; (d) generating a ligation product by ligating the first probe and the second probe: (e) hybridizing the ligation product to the capture domain; (f) generating a sequence that is complementary to the hybridized ligation product wherein the complementary sequence comprises one or more labeled nucleotides; and (h) detecting the signal, thereby identifying permeabilization conditions for the biological sample.

In another aspects, disclosed herein are methods for identifying permeabilization conditions for a biological sample on an array, the method comprising: (a) contacting the biological sample on the array, wherein the array comprises a plurality of capture probes, wherein a capture probe of the plurality comprises a capture domain; (b) permeabilizing the biological sample; (c) contacting a first probe and a second probe with the biological sample, wherein the first probe and the second probe each comprise sequences that are substantially complementary to sequences of a target nucleic acid, and wherein the second probe comprises a capture probe capture domain; (d) hybridizing the first probe and the second probe to the target nucleic acid; (e) generating a ligation product by ligating the first probe and the second probe; (f) hybridizing the ligation product to the capture domain; (g) generating a sequence that is complementary to the hybridized ligation product wherein the complementary sequence comprises one or more labeled nucleotides; and (h) detecting the signal, thereby identifying permeabilization conditions for the biological sample.

In some aspects, this disclosure features methods of testing a biological sample for efficacy of detection of a target nucleic acid, the method including: (a) contacting a biological sample on an array including a plurality of capture probes, wherein a capture probe of the plurality includes a capture domain; (b) contacting a first probe and a second probe with the biological sample, wherein the first probe and the second probe each include sequences that are substantially complementary to sequences of the target nucleic acid, and wherein the second probe includes a capture probe capture domain; (c) hybridizing a first probe and a second probe to the target nucleic acid; (d) generating a ligation product by ligating the first probe and the second probe; (e) releasing the ligation product from the target nucleic acid; (f) hybridizing the ligation product to the capture domain; (g) generating a sequence that is complementary to the bound ligation product wherein the complementary sequence includes one or more labeled nucleotides; and (h) detecting a signal corresponding to the ligation product on the substrate, thereby determining the efficacy of detection of the target nucleic acid.

In another aspect, this disclosure features methods for identifying permeabilization conditions for a biological sample on a spatial array, the method including: (a) contacting a biological sample on an array including a plurality of capture probes, wherein a capture probe of the plurality includes a capture domain; (b) contacting a first probe and a second probe with the biological sample, wherein the first probe and the second probe each include sequences that are substantially complementary to sequences of the target nucleic acid, and wherein the second probe includes a capture probe capture domain; (c) hybridizing a first probe and a second probe to the target nucleic acid; (d) generating a ligation product by ligating the first probe and the second probe; (e) releasing the ligation product from the target nucleic acid; (f) hybridizing the ligation product to the capture domain; (g) generating a sequence that is complementary to the bound ligation product wherein the complementary sequence includes one or more labeled nucleotides; and (h) detecting a signal corresponding to the ligation product on the substrate, thereby determining the identifying permeabilization conditions for the biological sample.

In some instances, correlating the intensity of the signal further determines degradation status of the target nucleic acid.

In some embodiments, the generating step includes a nucleic acid extension reaction. In some embodiments, the generating step includes extending the capture probe using the ligation product as a template, thereby generating a sequence that is complementary to the ligation product.

In some instances, the first probe and the second probe are substantially complementary to adjacent sequences of the target nucleic acid. In some instances, the first probe and the second probe hybridize to sequences that are not adjacent to each other on the target nucleic acid. In some instances, the first probe is extended with a DNA polymerase, thereby (i) filling in a gap between the first probe and the second probe and (ii) generating an extended first probe.

In some embodiments, the generating step includes contacting the ligation product with one or more of: an enzyme: a buffer; and a plurality of nucleotides (dNTPs), wherein the plurality of nucleotides includes one or more labelled nucleotides.

In some embodiments, the enzyme is a DNA polymerase. In some embodiments, the enzyme is a phi29 DNA polymerase. In some embodiments, the enzyme is a thermostable DNA polymerase.

In some embodiments, the enzyme is a reverse transcriptase enzyme. In some embodiments, the reverse transcriptase is selected from the group consisting of: avian myeloblastosis virus (AMV) reverse transcriptase, Moloney murine leukemia virus (M-MuLV or MMLV) reverse transcriptase, and HIV reverse transcriptase, or functional variants thereof. In some embodiments, the reverse transcriptase is an M-MLV reverse transcriptase enzyme.

In some embodiments, the plurality of nucleotides include one or more of a dATP, a dTTP, a dGTP, a dUTP and a dCTP.

In some embodiments, the labelled nucleotides include one or more of a labelled dATP, a labelled dTTP, a labeled dUTP, a labelled dGTP, or a labelled dCTP.

In some embodiments, labelled nucleotides include a label selected from the group consisting of: a radiolabel, a fluorescent label, a chemiluminescent label, a bioluminescent label, a calorimetric label, and a colorimetric label.

In some embodiments, the fluorescent label includes a fluorophore selected from the group consisting of: Cy3, Cy5, Cy5.5, Cy7, TAMRA, 5-ROX, TYE 653, HEX, TEX 615, TYE 665, and TYE 705.

In some instances, the level of the RNA is transcribed from a housekeeping gene. In some embodiments, the target nucleic acid includes RNA. In some embodiments, the level of the RNA is highly abundant in the biological sample as compared to the level of other RNA molecules in the biological sample. In some embodiments, the RNA is a ribosomal RNA (rRNA). In some embodiments, the rRNA is selected from the group consisting of 18S, 28S, 5.8S, 5S, 12S, 16S, and 23S. In some embodiments, the rRNA is 18S.

In some embodiments, the first probe includes a sequence that is substantially complementary to a first sequence of the rRNA.

In some embodiments, the second probe includes a sequence that is substantially complementary to a second sequence of the rRNA.

In some embodiments, the first sequence and the second sequence are adjacent in the rRNA.

In some embodiments, the first probe includes a sequence that is at least 80% identical to SEQ ID NO: 1. In some embodiments, the first probe includes a sequence of SEQ ID NO: 1.

In some embodiments, the second probe includes a sequence that is at least 80% identical to SEQ ID NO: 2. In some embodiments, the second probe includes a sequence of SEQ ID NO: 2.

In some embodiments, the first probe further includes a functional sequence, wherein the functional sequence is a primer sequence.

In some embodiments, the biological sample is contacted with the first probe and the second probe at a total concentration of about 25 nM to about 2500 nM. In some embodiments, the biological sample is contacted with the first probe and the second probe at a total concentration of about 100 nM to about 2000 nM. In some embodiments, the biological sample is contacted with the first probe and the second probe at a total concentration of about 2000 nM.

In some embodiments, the first probe and/or the second probe is a DNA probe.

In some embodiments, the method further includes contacting the biological sample with one or more additional probe pairs, wherein a probe pair includes an additional first probe and an additional second probe.

In some embodiments, each of the one or more additional probe pairs includes (i) a first sequence that is substantially complementary to a sequence of the rRNA, and (ii) a second sequence that is substantially complementary to a sequence of the rRNA, wherein the first sequence and the second sequence are adjacent in the IRNA.

In some embodiments, each of the one or more additional probe pairs includes (i) a first sequence that is substantially complementary to a sequence of a second rRNA, and (ii) a second sequence that is substantially complementary to a sequence of a second rRNA, wherein the first sequence and the second sequence are adjacent in the second rRNA.

In some embodiments, each of the one or more additional probe pairs includes (i) a first sequence that is substantially complementary to a sequence of a second target nucleic acid, and (ii) a second sequence that is substantially complementary to a sequence of a second target nucleic acid.

In some embodiments, generating a ligation product includes ligating the first probe to the second probe, wherein the enzymatic ligation utilizes a ligase. In some embodiments, the ligase is one or more of a T4 RNA ligase (Rnl2), a PBCV-1 ligase, a ligase from a Chlorella virus, a ligase that ligates two DNA strands that are adjacently positioned on a RNA molecule, a single stranded DNA ligase, or a T4 DNA ligase.

In some embodiments, the method further includes permeabilizing the biological sample. In some embodiments, permeabilizing includes contacting the biological sample with a permeabilization agent. In some embodiments, permeabilization agent is selected from an organic solvent, a detergent, and an enzyme, or a combination thereof. In some embodiments, the permeabilization agent is selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, a nonionic surfactant (e.g. Triton X-100™), and polyoxyethylene sorbitol (e.g., Tween-20™). In some embodiments, permeabilizing the biological sample includes an endopeptidase. In some embodiments, the endopeptidase is pepsin or proteinase K.

In some embodiments, the method further includes providing a capture probe capture domain blocking moiety that interacts with the capture probe capture domain.

In some embodiments, the method further includes releasing the capture probe capture domain blocking moiety from the capture probe capture domain prior to the step of hybridizing the ligation product to the capture domain.

In some embodiments, the capture probe capture domain includes a poly-adenylated (poly(A)) sequence or a complement thereof.

In some embodiments, the capture probe capture domain blocking moiety includes a poly-uridine sequence, a poly-thymidine sequence, or both.

In some embodiments, releasing the poly-uridine sequence or poly-thymidine sequence from the poly(A) sequence includes denaturing the ligation product or contacting the ligation product with an endonuclease, exonuclease or ribonuclease.

In some embodiments, the capture probe capture domain includes a sequence that is complementary to all or a portion of the capture domain of the capture probe.

In some embodiments, the capture probe capture domain blocking moiety is a DNA probe.

In some embodiments, including removing the biological sample from the substrate. In some embodiments, the removing step is performed prior to the step of hybridizing the ligation product to the capture domain. In some embodiments, the removing step is performed prior to the step of generating a sequence that is complementary to the hybridized ligation product.

In some embodiments, the signal corresponding to the bound ligation product includes the signal from the labeled dNTPs.

In some embodiments, the detecting step includes obtaining an image corresponding to the signal corresponding to the bound ligation product on the substrate.

In some embodiments, the method further includes registering image coordinates to a fiducial marker.

In some embodiments, the method further includes analyzing a signal corresponding to the bound ligation product on the substrate.

In some embodiments, the further includes identifying, based on the signal analysis, the quality of the biological sample.

In some embodiments, the biological sample is a tissue sample. In some embodiments, the tissue sample is a formalin-fixed, paraffin-embedded (FFPE) tissue sample. In some embodiments, the tissue sample is the FFPE tissue sample, and the tissue sample is decrosslinked.

In some embodiments, the biological sample was previously stained. In some embodiments, the biological sample was previously stained using immunofluorescence or immunohistochemistry. In some embodiments, the biological sample was previously stained using hematoxylin and eosin.

In some embodiments, the method further includes selecting, based on the detected signal, a second tissue section of the biological sample for the detection of one or more additional target nucleic acids.

In some embodiments, the second tissue section is a serial section of the biological sample.

In some embodiments, the method further includes analyzing the second portion of the biological sample for the detection of one or more additional target nucleic acids, wherein analyzing the second portion includes determining a location and abundance of one or more target nucleic acids in the second portion of the biological sample.

In some embodiments, the steps of analyzing include: (a) providing the second tissue section of the biological sample on a second array including a second plurality of second capture probes, wherein a second capture probe of the second plurality of second capture probes includes: (i) a spatial barcode and (ii) a second capture domain; (b) hybridizing a second plurality of probes with the biological sample, wherein a first probe of the second plurality of probes and a second probe of the second plurality of probes each include sequences that are substantially complementary to sequences of the target nucleic acid, and wherein the second probe of the second plurality of probes includes a second capture domain; (c) generating a second ligation product by ligating the first probe of the second plurality of probes and the second probe of the second plurality of probes; (d) releasing the second ligation product from the target nucleic acid: (e) hybridizing the second ligation product to the second capture domain; and (f) determining (i) all or a part of the sequence of the second ligation product bound to the capture domain, or a complement thereof, and (ii) the sequence of the spatial barcode, or a complement thereof, and using the determined sequence of (i) and (ii) to identify the location of the target nucleic acid in the biological sample.

In some embodiments, the methods further include a releasing step that comprises removing the ligation product from the nucleic acid prior to hybridizing the ligation product to the capture domain. In some instances, the releasing of (i) the ligation product from the target nucleic acid or (ii) the capture probe capture domain blocking moiety from the capture domain binding domain, comprises contacting the ligated probe with an endoribonuclease. In some instances, the endoribonuclease is one or more of RNase H, RNase A, RNase C, or RNase I. In some instances, the endoribonuclease is RNase H. In some instances, the RNase H comprises RNase H1, RNase H2, or RNase H1 and RNase H2.

In some instances, permeabilizing the biological sample occurs before contacting a first probe and a second probe with the biological sample. In some instances, permeabilizing the biological sample occurs before releasing the ligation product from the target nucleic acid. In some instances, permeabilizing the biological sample occurs after releasing the ligation product from the target nucleic acid.

In another aspect, this disclosure features kits including: (a) a substrate including a plurality of capture probes including a spatial barcode and a capture domain; (b) a system including: a plurality of first probes and second probes, wherein a first probe and a second probe each includes sequences that are substantially complementary to an IRNA, and wherein the second probe includes a capture binding domain; (c) a plurality of enzymes including a ribonuclease, a ligase, and a polymerase (d) a plurality of labelled dNTPS; and (e) instructions for performing any of the methods described herein.

In another aspect, this disclosure features kits including: (a) an array including a plurality of capture probes; (b) a plurality of probes including a first probe and a second probe, wherein the first probe and the second probe are substantially complementary to adjacent sequences of a rRNA, wherein the second probe includes a capture probe capture domain that is capable of binding to a capture domain of the capture probe: an enzyme including (c) a plurality of enzymes including a ribonuclease, a ligase, and a polymerase; (d) a plurality of labelled dNTPs; and (e) instructions for performing any of the methods described herein. In some embodiments of any of the kits described herein, the rRNA is 18S.

In some embodiments of any of the kits described herein, the first probe includes a sequence that is at least 80% identical to SEQ ID NO: 1.

In some embodiments of any of the kits described herein, the second probe includes a sequence that is at least 80% identical to SEQ ID NO: 2.

In another aspect, this disclosure features compositions including a spatial array including capture probes, wherein the capture probes include a capture domain, a biological sample on the spatial array wherein the biological sample includes a plurality of rRNA of interest, a first probe and a second probe hybridized to the rRNA of interest and ligated together, wherein the first probe and the second probe each include a sequence that is substantially complementary to adjacent sequences of the rRNA and wherein one of the first probe or the second probe includes a capture probe capture domain.

In some embodiments, the composition further includes an RNase H enzyme.

In some embodiments, the composition further includes an RNase A enzyme.

In some embodiments, the composition further includes a ligase. In some embodiments of any of the compositions described herein, one of the first probe or the second probe includes a functional sequence.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow using targeted nucleic acid capture.

FIG. 2 is a schematic diagram showing an example of a barcoded capture probe, as described herein.

FIG. 3 is a schematic illustrating a cleavable capture probe, wherein the cleaved capture probe can enter into a non-permeabilized cell and bind to target nucleic acids within the sample.

FIG. 4 is a schematic diagram of an exemplary multiplexed spatially-barcoded feature.

FIG. 5 is a schematic showing the exemplary arrangement of barcoded features within an array.

FIG. 6 is a schematic diagram showing an exemplary workflow for templated ligation.

FIG. 7 is an exemplary workflow for a method including labelling a ligation product with labelled nucleotides.

FIG. 8 is a schematic diagram showing an exemplary workflow for capturing a ligation product on a substrate that includes capture probes.

FIG. 9 shows from left to right, experimental conditions for mouse brain tissue sections, H&E stained mouse brain tissue sections and fluorescent image results from the experimental conditions for the tissue sections.

FIG. 10 shows from left to right, experimental conditions for mouse brain tissue sections, H&E stained mouse brain tissue sections and fluorescent image results from the experimental conditions for the tissue sections.

FIG. 11 shows from left to right, experimental conditions for mouse brain tissue sections, H&E stained mouse brain tissue sections and fluorescent image results from the experimental conditions for the tissue sections.

FIG. 12 shows from left to right, experimental conditions for mouse brain tissue sections, H&E stained mouse brain tissue sections and fluorescent image results from the experimental conditions for the tissue sections.

FIG. 13 shows from left to right, experimental conditions for different human and mouse tissue sections, H&E stained sections and fluorescent image results from the experimental conditions for the tissue sections.

FIG. 14 shows from left to right, experimental conditions for mouse brain tissue sections, H&E stained mouse brain tissue sections and fluorescent image results from the experimental conditions for the tissue sections.

FIG. 15 shows from left to right, experimental conditions for mouse brain tissue sections, H&E stained mouse brain tissue sections and fluorescent image results from the experimental conditions for the tissue sections.

FIG. 16 shows from left to right, experimental conditions for mouse brain tissue sections, H&E stained mouse brain tissue sections and fluorescent image results from the experimental conditions for the tissue sections.

FIGS. 17A-17E shows human breast cancer tissue sections from a human breast cancer tissue sample. FIG. 17A shows an H&E stain of a first tissue section. FIG. 17B shows a fluorescently labeled nucleotide that was incorporated during second strand synthesis from a first tissue section, FIG. 17C shows an H&E stain of a second serial tissue section. FIG. 17D shows a spatial gene expression map from the second tissue section. FIG. 17E shows a log 10 UMI count heat map from the second tissue section.

FIGS. 18A-18E show mouse kidney tissue sections from a mouse tissue sample. FIG. 18A shows H&E stain from a first tissue section. FIG. 18B shows a fluorescently labeled nucleotide that was incorporated during second strand synthesis in a first tissue section. FIG. 18C shows H&E stain from a second serial tissue section. FIG. 18D shows a spatial gene expression map of the second tissue section. FIG. 18E shows a log 10 UMI heat map of the second tissue section.

FIGS. 19A-19E are mouse spleen tissue sections from a mouse spleen sample. FIG. 19A shows H&E stain from a first tissue section. FIG. 19B shows a fluorescently labeled nucleotide that was incorporated during second strand synthesis from the first tissue section.

FIG. 19C shows H&E stain of a second serial tissue section. FIG. 19D shows a spatial gene expression map of the second tissue section. FIG. 19E shows a log 10 UMI heat map of the second tissue section.

### DETAILED DESCRIPTION

Targeted RNA capture is an attractive alternative to poly(A) mRNA capture to interrogate spatial gene expression in FFPE tissue. Compared to poly(A) mRNA capture, targeted RNA capture is less affected by RNA degradation associated with FFPE fixation than methods dependent on oligo-dT capture and reverse transcription of mRNA: allows for sensitive measurement of specific genes of interest that otherwise might be missed with a whole transcriptomic approach; and is scalable, with demonstrated probes targeting a large fraction of the transcriptome.

Spatial analysis methodologies and compositions described herein can provide a vast amount of nucleic acid and/or expression data for a variety of nucleic acids within a biological sample at high spatial resolution, while retaining native spatial context. Spatial analysis methods and compositions can include, e.g., the use of a capture probe including a spatial barcode (e.g., a nucleic acid sequence that provides information as to the location or position of a nucleic acid within a cell or a tissue sample (e.g., mammalian cell or a mammalian tissue sample) and a capture domain that is capable of hybridizing to a nucleic acid by and/or present in a cell. Spatial analysis methods and compositions can also include the use of a capture probe having a capture domain that captures an intermediate agent for indirect detection of a nucleic acid. For example, the intermediate agent can include a nucleic acid sequence (e.g., a barcode) associated with the intermediate agent. Detection of the intermediate agent is therefore indicative of the nucleic acid in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3):442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018: the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest. As used herein, “analyte” can refer to a target nucleic acid.

Analytes can be broadly classified into one of two groups: nucleic acid analytes (e.g., target nucleic acids), and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in WO 2020/176788 and/or U.S. Patent Application Publication No. 2020/0277663.

In some embodiments, a biological sample is permeabilized with one or more permeabilization reagents. For example, permeabilization of a biological sample can facilitate target nucleic acid capture. Exemplary permeabilization agents and conditions are described in WO 2020/176788 and/or U.S. Patent Application Publication No. 2020/0277663.

Array-based spatial analysis methods involve the transfer of one or more target nucleic acids from a biological sample to an array of features on a substrate, where each feature is associated with a unique spatial location on the array. Subsequent analysis of the transferred target nucleic acids includes determining the identity of the target nucleic acids and the spatial location of the target nucleic acids within the biological sample. The spatial location of a target nucleic acid within the biological sample is determined based on the feature to which the target nucleic acid is bound (e.g., directly or indirectly) on the array, and the feature's relative spatial location within the array.

A “capture probe” refers to any molecule capable of capturing (directly or indirectly) and/or labelling a target nucleic acid (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., WO 2020/176788 and/or U.S. Patent Application Publication No. 2020/0277663. Generation of capture probes can be achieved by any appropriate method, including those described in WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in WO 2020/176788 and/or U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). In some instances, the spatially-barcoded array populated with capture probes (as described further herein) is contacted with a biological sample, and the biological sample is permeabilized, allowing the analyte to migrate away from the sample and toward the array. The analyte interacts with a capture probe on the spatially-barcoded array. Another method is to cleave spatially-barcoded capture probes from an array and promote the spatially-barcoded capture probes towards and/or into or onto the biological sample. In some instances, the spatially-barcoded array populated with capture probes (as described further herein) can be contacted with a sample. The spatially-barcoded capture probes are cleaved and then interact with cells within the provided biological sample. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell.

In some instances, sample preparation may include placing the sample on a slide, fixing the sample, and/or staining the biological sample for imaging. The stained sample can be then imaged on the array using both brightfield (to image the sample hematoxylin and eosin stain) and/or fluorescence (to image features) modalities. Optionally, the sample can be destained prior to permeabilization. In some embodiments, analytes, or analyte derived products (e.g., ligation products) are then released from the sample and capture probes forming the spatially-barcoded array hybridize or bind the released analytes or analyte derived products (e.g., ligation products). The sample is then removed from the array and the capture probes cleaved from the array. The biological sample and array are then optionally imaged a second time in one or both modalities while the analytes are reverse transcribed into cDNA, and an amplicon library is prepared and sequenced. Images are then spatially-overlaid in order to correlate spatially-identified biological sample information. When the sample and array are not imaged a second time, a spot coordinate file is supplied instead. The spot coordinate file replaces the second imaging step. Further, amplicon library preparation can be performed with a unique PCR adapter and sequenced.

In some instances, disclosed is another exemplary workflow that utilizes a spatially-barcoded array on a substrate, where spatially-barcoded capture probes are clustered at areas called features. The spatially-barcoded capture probes can include a cleavage domain, one or more functional domains, a spatial barcode, a unique molecular identifier, and a capture domain. The spatially-barcoded capture probes can also include a 5′ end modification for reversible attachment to the substrate. The spatially-barcoded array is contacted with a biological sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes or analyte derived products (e.g., ligation products) migrate toward the spatially-barcoded capture array using any number of techniques disclosed herein. For example, analyte or analyte derived product (e.g., ligation products) migration can occur using a diffusion-resistant medium lid and passive migration. As another example, analyte or analyte derived product (e.g., ligation products) migration can be active migration, using an electrophoretic transfer system, for example. Once the analytes are in close proximity to the spatially-barcoded capture probes, the capture probes can hybridize or otherwise bind a target analyte. The biological sample can be optionally removed from the array.

The capture probes can be optionally cleaved from the array, and the captured analytes or analyte derived products (e.g., ligation products) can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme in a template independent manner. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be amplified using PCR, where the forward and reverse primers flank the spatial barcode and analyte regions of interest, generating a library associated with a particular spatial barcode. In some embodiments, the library preparation can be quantitated and/or quality controlled to verify the success of the library preparation steps. In some embodiments, the cDNA comprises a sequencing by synthesis (SBS) primer sequence. The library amplicons are sequenced and analyzed to decode spatial information.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some instances, the capture probe can include functional sequences that are useful for subsequent processing. In some instances, a capture probe can be reversibly attached to a substrate via a linker. The capture probe can include one or more functional sequences, which can include a sequencer specific flow cell attachment sequence, e.g., a P5 or P7 sequence, as well as functional sequence, which can include sequencing primer sequences, e.g., a R1 primer binding site, a R2 primer binding site. In some embodiments, sequence is a P7 sequence and sequence is a R2 primer binding site. A capture probe can additionally include a spatial barcode and/or unique molecular identifier and a capture domain. The different sequences of the capture probe need not be in the sequential manner as depicted in this example, however the capture domain should be placed in a location on the barcode wherein analyte capture and extension of the capture domain to create a copy of the analyte can occur.

In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

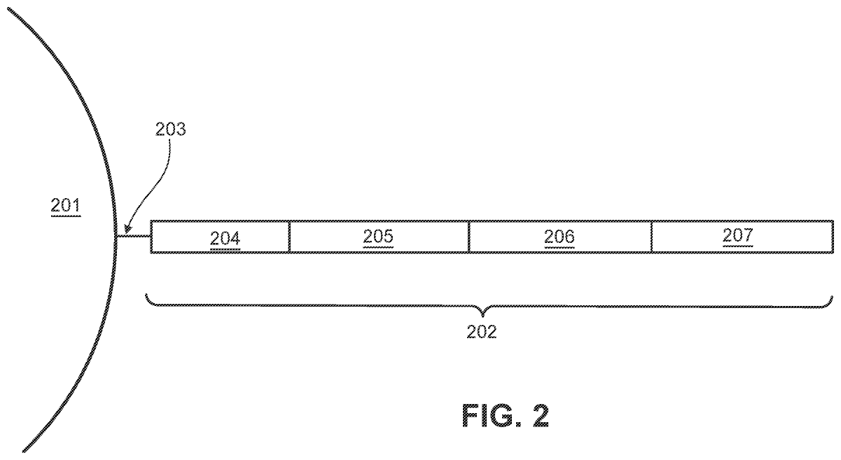

FIG. 2 is a schematic diagram showing an exemplary capture probe, as described herein. As shown, the capture probe 202 is optionally coupled to a feature 202 by a cleavage domain 203, such as a disulfide linker. The capture probe can include a functional sequence 204 that are useful for subsequent processing. The functional sequence 204 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 205. The capture probe can also include a unique molecular identifier (UMI) sequence 206. While FIG. 2 shows the spatial barcode 205 as being located upstream (5′) of UMI sequence 206, it is to be understood that capture probes wherein UMI sequence 206 is located upstream (5′) of the spatial barcode 205 is also suitable for use in any of the methods described herein. The capture probe can also include a capture domain 207 to facilitate capture of a target analyte. In some embodiments, the capture probe comprises one or more additional functional sequences that can be located, for example between the spatial barcode 205 and the UMI sequence 206, between the UMI sequence 206 and the capture domain 207, or following the capture domain 207. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

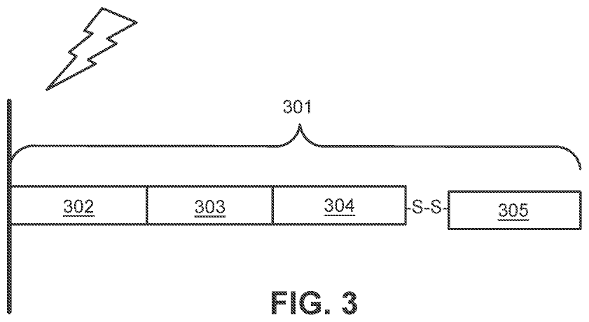

In some cases, capture probes are introduced into the cell using a cell-penetrating peptide. FIG. 3 is a schematic illustrating a cleavable capture probe that includes a cell-penetrating peptide, wherein the cleaved capture probe can enter into a non-permeabilized cell and bind to analytes within the sample. The capture probe 301 contains a cleavage domain 302, a cell penetrating peptide 303, a reporter molecule 304, and a disulfide bond (—S—S—). 305 represents all other parts of a capture probe, for example a spatial barcode and a capture domain.

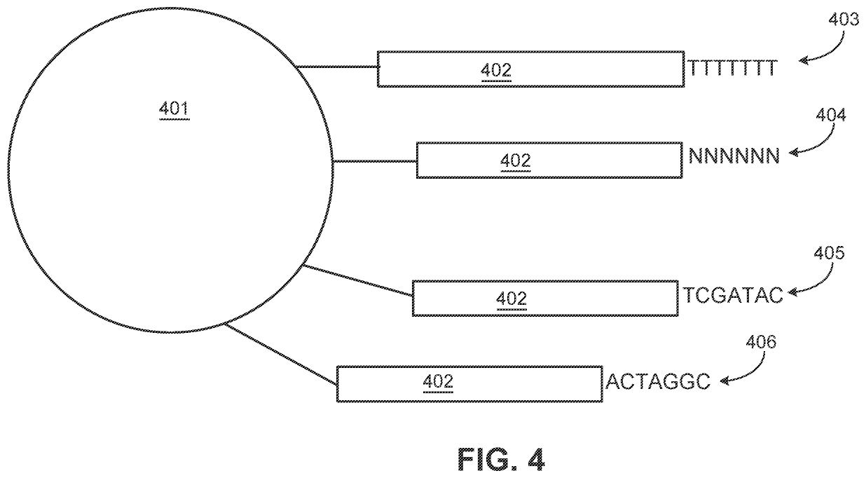

In some instances, the disclosure provides multiplexed spatially-barcoded features. FIG. 4 is a schematic diagram of an exemplary multiplexed spatially-barcoded feature. In FIG. 4, the feature 401 (e.g., a bead, a location on a slide or other substrate, a well on a slide or other substrate, a partition on a slide or other substrate, etc.) can be coupled to spatially-barcoded capture probes, wherein the spatially-barcoded probes of a particular feature can possess the same spatial barcode, but have different capture domains designed to associate the spatial barcode of the feature with more than one target nucleic acid. For example, a feature may be coupled to four different types of spatially-barcoded capture probes, each type of spatially-barcoded capture probe possessing the spatial barcode 402. One type of capture probe associated with the feature includes the spatial barcode 402 in combination with a poly(T) capture domain 403, designed to capture target nucleic acids (e.g., mRNA). A second type of capture probe associated with the feature includes the spatial barcode 402 in combination with a random N-mer capture domain 404 for gDNA analysis. A third type of capture probe associated with the feature includes the spatial barcode 402 in combination with a capture domain complementary to the analyte capture agent of interest 405. A fourth type of capture probe associated with the feature includes the spatial barcode 402 in combination with a capture probe that can specifically bind a nucleic acid molecule 406 that can function in a CRISPR assay (e.g., CRISPR/Cas9). While only four different capture probe-barcoded constructs are shown in FIG. 4, capture-probe barcoded constructs can be tailored for analyses of any given analyte associated with a nucleic acid and capable of binding with such a construct. For example, the schemes shown in FIG. 4 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq. DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

Additional features of capture probes are described in WO 2020/176788 and/or U.S. Patent Application Publication No. 2020/0277663, each of which is incorporated by reference in its entirety. Generation of capture probes can be achieved by any appropriate method, including those described in WO 2020/176788 and/or U.S. Patent Application Publication No. 2020/0277663, each of which is incorporated by reference in its entirety.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in WO 2020/176788 and/or U.S. Patent Application Publication No. 2020/0277663. Some quality control measures are described in WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder: identification of a candidate drug target for treatment of a disease or disorder: identification (e.g., diagnosis) of a subject as having a disease or disorder: identification of stage and/or prognosis of a disease or disorder in a subject; identification of a subject as having an increased likelihood of developing a disease or disorder: monitoring of progression of a disease or disorder in a subject: determination of efficacy of a treatment of a disease or disorder in a subject: identification of a patient subpopulation for which a treatment is effective for a disease or disorder: modification of a treatment of a subject with a disease or disorder: selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue): identification of multiple analyte types in close proximity (e.g., nearest neighbor analysis); determination of up- and/or down-regulated genes and/or proteins in diseased tissue: characterization of tumor microenvironments: characterization of tumor immune responses: characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in WO 2020/176788 and/or U.S. Patent Application Publication No. 2020/0277663. Exemplary features and geometric attributes of an array can be found in WO 2020/176788 and/or U.S. Patent Application Publication No. 2020/0277663.

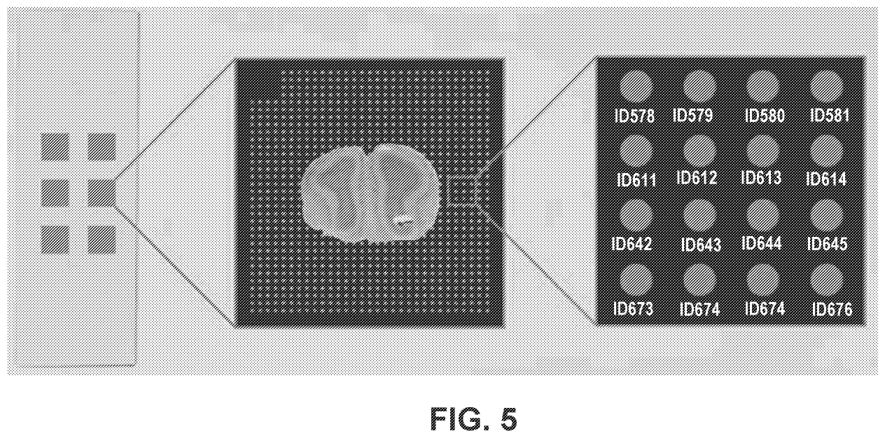

FIG. 5 depicts an exemplary arrangement of barcoded features within an array. From left to right, FIG. 5 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (labelled as ID578, ID579, ID560, etc.).

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells, areas on a substrate) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Templated ligation, or RTL, is disclosed in US 2021/0348221, US 2021/0285046, and WO 2021/133849, each of which is incorporated by reference in its entirety. Additional methods of RTL have been described previously. See, e.g., Credle et al., Nucleic Acids Res. 2017 Aug 21:45(14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., PBCV-1 ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in WO 2020/176788 and/or U.S. Patent Application Publication No. 2020/0277663, or any of one or more of the devices or methods described in WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### I. RNA Capture Using Labeled RNA-Templated Ligation

### (a) Introduction

Targeted RNA capture is a method that examines a more limited number of nucleic acids than is typically evaluated in whole genome or whole exome sequencing. In a non-limiting example, capturing a derivative of a nucleic acid (e.g., a ligation product) provides enhanced specificity with respect to detection of a nucleic acid. This is because at least two probes specific for a target are required to hybridize to the target in order to facilitate ligation and ultimate capture of the nucleic acid. In order to assess whether a biological sample is suitable for targeted RNA capture, the biological sample can be assessed prior to targeted RNA capture. The methods described herein provide methods for assessing efficiency of targeted RNA capture.

In particular, the methods disclosed examine the capture of a highly abundant RNA molecule in a biological sample. For instances, the methods disclosed herein determine the abundance of a ribosomal RNA (e.g., 18S) in a biological sample. Based on the detection readout of the abundant RNA, it can be determined whether a sample is a good candidate for examination of abundance and location of a larger cohort of RNA molecules (e.g., whole or partial exome)

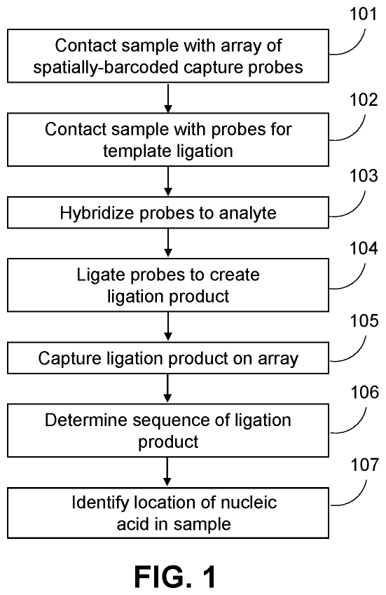

Referring to FIG. 1, the methods disclosed herein identify conditions that will help determine whether the biological sample is likely to yield data with regards to abundance and location of a nucleic acid in a biological sample using RNA template ligation. FIG. 1 demonstrates a workflow that could be followed once it was determined that a biological sample would yield useful spatial gene expression data using the RTL workflow. The method 101 of RNA template ligation includes contacting a biological sample with an array of spatially-barcoded capture probes. In some instances, the array is on a substrate and the array includes a plurality of capture probes, wherein a capture probe of the plurality includes: (i) a spatial barcode and (ii) a capture domain. After placing the biological sample on the array, the biological sample 102 is contacted with a first probe and a second probe (e.g., probes with sequences complementary to a highly abundant RNA molecule such as 18S), wherein the first probe and the second probe each include one or more sequences that are substantially complementary to sequences of the nucleic acid, and wherein the second probe includes a capture probe capture domain: the first probe and the second probe 103 hybridize to complementary sequences in the nucleic acid. After hybridization a ligation product comprising the first probe and the second probe 104 is generated, and the ligation product is released from the target nucleic acid. The liberated ligation product 105 can hybridize to the capture domain of a probe on the array. After capture, (i) all or a part of the sequence of the ligation product specifically hybridized to the capture domain, or a complement thereof, and (ii) the sequence of the spatial barcode, or a complement thereof 106 can be determined, and then one can use the determined sequence of (i) and (ii) 107 to identify the location of the target nucleic acid in the biological sample.

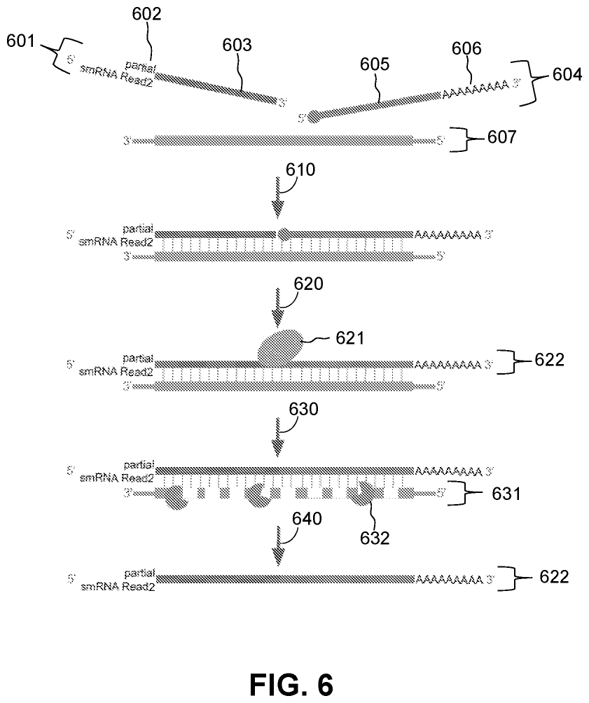

A non-limiting example of the targeted RNA capture methods disclosed herein is depicted in FIG. 6. After a biological sample is contacted with a substrate including a plurality of capture probes and contacted with (a) a first probe 601 having a target-hybridization sequence 603 and a primer sequence 602 and (b) a second probe 604 having a target-hybridization sequence 605 and a capture domain (e.g., a poly-A sequence) 606, the first probe 601 and a second probe 604 hybridize 610 to a target nucleic acid 607 (e.g., a highly abundant RNA molecule (e.g., an rRNA)). A ligase 621 ligates 620 the first probe to the second probe thereby generating a ligation product 622. The ligation product is released 630 from the target nucleic acid 631 (e.g., a highly abundant RNA molecule (e.g., an rRNA)) by digesting the nucleic acid using an endoribonuclease 632. The sample is permeabilized 640 and the ligation product 622 is able to hybridize to a capture probe on the substrate.

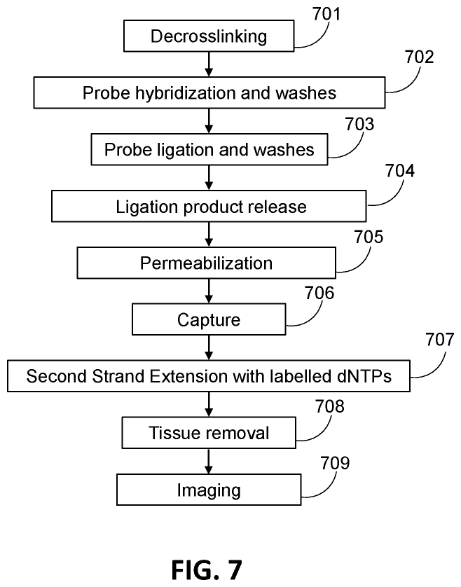

FIG. 7, demonstrates a general workflow for determining whether nucleic acids in a biological sample are of a quality where RTL gene expression analysis data could be obtained. A biological sample is decrosslinked and the biological sample is assessed for efficacy of detection of the ligation product. Briefly, a FFPE biological sample on an array is decrosslinked 701 and probes are added to the sample and hybridized to a nucleic acid 702. Probes are ligated 703 and released using an endonuclease such as RNAse H 704. The biological sample is permeabilized 705 and the ligation product hybridizes to a capture probe on the array 706. In some instances, the capture probe does not include a spatial barcode. The capture probe is extended 707 using the ligation product as a template thereby generating a sequence that is complementary to the ligation product and includes Cy3-dCTP nucleotides. The biological sample is removed from the array 708 and the array is imaged 709. The signal corresponding to the bound ligation product that includes Cy3-dCTPs from the image is used to identify the quality of the biological sample. Practicing the method of FIG. 7 to determine the quality of the biological sample, the capture probe does not need to include a spatial barcode or a UMI, as fluorescence is used as the direct measure of whether second strand synthesis has occurred. Therefore, while a spatial barcode. UMI and other functional sequences can be present, they are not required when practicing the methods described herein to determine the quality of target nucleic acids present in a biological sample.

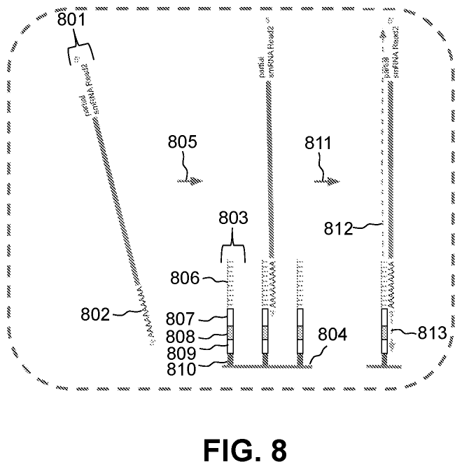

In one embodiment, the method as described in FIG. 8 can be generally followed when ascertaining the quality of target nucleic acids in a biological sample for further spatial gene expression analysis. As shown in FIG. 8, the ligation product 801 includes a capture probe capture domain 802, which can bind to a capture probe 803 (e.g., a capture probe immobilized, directly or indirectly, on a substrate 804). In some embodiments, methods provided herein include contacting 805 a biological sample with a substrate 804, wherein the capture probe 803 is affixed to the substrate (e.g., immobilized to the substrate, directly or indirectly). In some embodiments, the capture probe capture domain 802 of the ligated product specifically hybridize to the capture domain 806. In some instances, the capture probe can also include a unique molecular identifier (UMI) 807, a spatial barcode 808, a functional sequence 809, and a cleavage domain 810. In some instances, the capture probe includes a capture probe, but does not include a unique molecular identifier (UMI) 807, a spatial barcode 808, a functional sequence 809, and a cleavage domain 810.

Practicing the method of FIG. 8 to determine the quality of the nucleic acids in a biological sample, the capture probe does not need to include a spatial barcode or a UMI, as fluorescence is used as the direct measure of whether second strand synthesis has occurred. Therefore, while a spatial barcode. UMI and other functional sequences can be present, they are not required when practicing the methods described herein to determine the quality of nucleic acids in a biological sample.

In some embodiments methods provided herein include permeabilization of the biological sample such that the capture probe can more easily hybridize to the captured ligated probe (i.e., compared to no permeabilization). In some embodiments, reverse transcription (RT) reagents can be added to permeabilized biological samples. Incubation with extension reagents and labelled nucleotides 814 can be used to extend the capture probes 811 to produce a sequence 812 that is complementary to the ligation product 801 and includes one or more labelled nucleotides spatially-barcoded full-length cDNA 812 and 813 from the captured target nucleic acids (e.g., polyadenylated mRNA) that include one or more labelled nucleotide. In some embodiments, second strand reagents (e.g., second strand primers, enzymes) can be added to the biological sample on the slide to initiate second strand synthesis. In some embodiments, incubation with the second strand (SS) extension reagents and labelled nucleotides can extend the capture probes to produce a sequence that is complementary to the ligation product and includes one or more labelled nucleotides.

Also provided herein are methods of testing a biological sample for conditions that improve the efficacy of detection of a target nucleic acid, the method including: (a) contacting a biological sample on an array including a plurality of capture probes, wherein a capture probe of the plurality includes a capture domain; (b) contacting a first probe and a second probe with the biological sample, wherein the first probe and the second probe each include sequences that are substantially complementary to sequences of a target nucleic acid, and wherein the second probe includes a capture probe capture domain; (c) hybridizing a first probe and a second probe to the target nucleic acid; (d) generating a ligation product by ligating the first probe and the second probe: (e) releasing the ligation product from the target nucleic acid: (f) hybridizing the ligation product to the capture domain: (g) generating a sequence that is complementary the bound ligation product; and (h) detecting a signal corresponding to the ligation product on the substrate, thereby determining the conditions that improve the efficacy of detection of a target nucleic acid. As previously stated, spatial barcodes, UMIs and other functional capture probe sequences which are found when practicing spatial gene expression analysis are not necessary when determining biological sample quality or conditions that improve efficacy of detection of a target nucleic acid as described here, but they can be present.

Also provided herein are methods of identifying permeabilization conditions for a biological sample on a spatial array, the method including: (a) contacting a biological sample on an array including a plurality of capture probes, wherein a capture probe of the plurality includes a capture domain; (b) contacting a first probe and a second probe with the biological sample, wherein the first probe and the second probe each include sequences that are substantially complementary to sequences of a target nucleic acid, and wherein the second probe includes a capture probe capture domain; (c) hybridizing a first probe and a second probe to the target nucleic acid; (d) generating a ligation product by ligating the first probe and the second probe: (e) releasing the ligation product from the target nucleic acid: (f) hybridizing the ligation product to the capture domain: (g) generating a sequence that is complementary the hybridized ligation product; and (h) detecting a signal corresponding to the ligation product on the substrate, thereby determining the optimal permeabilization conditions for a biological sample on a spatial array.

Additional embodiments of the disclosure are provided herein.

### (b) Probes for Templated Ligation

The methods provided herein utilize probe pairs (or sets: the terms are interchangeable). In some instances, the probe pairs are designed so that each probe hybridizes to a sequence in a target nucleic acid that is specific to the target nucleic acid (e.g., compared to the entire genome). That is, in some instances, a single probe pair can be specific to a single target nucleic acid.

In other embodiments, probes can be designed so that one of the probes of a pair is a probe that hybridizes to a specific sequence. Then, the other probe can be designed to detect a mutation of interest. Accordingly, in some instances, multiple second probes can be designed and can vary so that each hybridizes to a specific sequence. For example, one second probe can be designed to hybridize to a wild-type sequence, and another second probe can be designed to detect a mutated sequence. Thus, in some instances, a probe set can include one first probe and two second probes (or vice versa).

On the other hand, in some instances, probes can be designed so that they cover conserved regions of a target nucleic acid. Thus, in some instances, a probe (or probe pair can hybridize to similar nucleic acids in a biological sample (e.g., to detect conserved or similar sequences) or in different biological samples (e.g., across different species).

In some instances, more than one probe oligonucleotide pair (e.g., a probe pair including a first probe and a second probe) is designed to cover one target nucleic acid. For example, at least two, three, four, five, six, seven, eight, nine, ten, or more probe sets can be used to hybridize to a single target nucleic acid. Factors to consider when designing probes is presence of variants (e.g., SNPs, mutations) or multiple isoforms expressed by a single gene. In some instances, the probe oligonucleotide pair does not hybridize to the entire nucleic acid, but instead the probe oligonucleotide pair hybridizes to a portion of the entire nucleic acid.

In some instances, about 100, 200, 300, 400, 500, 600, 700, 800, 900, 1000, 2000, 3000, 4000, 5000, 10,000, 15,000, 20,000, or more probe oligonucleotides pair (e.g., a probe pair including a first probe and a second probe) are used in the methods described herein. In some instances, about 20,000 probe oligonucleotides pair are used in the methods described herein.

In some instances, RNA capture is targeted RNA capture. Targeted RNA capture using the methods disclosed herein allows for examination of a subset of RNA sequences from the entire transcriptome or whole genome. In some embodiments, the subset of sequences includes an individual target RNA. In some embodiments, the subset of sequences includes two or more targeted RNAs. In some embodiments, the subset of nucleic acids includes one or more mRNAs transcribed by one or more targeted genes. In some embodiments, the subset of nucleic acids includes one or more mRNA splice variants of one or more targeted genes. In some embodiments, the subset of nucleic acids includes non-polyadenylated RNAs in a biological sample. In some embodiments, the subset of nucleic acids includes detection of mRNAs having one or more single nucleotide polymorphisms (SNPs) in a biological sample.

As used herein, the term “highly abundant” refers to a nucleic acid that is present in the biological sample at a level that is detectable using the methods described herein. In some instances, a highly abundant nucleic acid comprises sequences from ribosomal RNA or transfer RNA, or mRNA for highly expressed genes or genes that are considered to be expressed in almost all cells of an organism, such as housekeeping genes. In some embodiments, the level of a nucleic acid is highly abundant in the biological sample as compared to the level of other nucleic acid molecules in the biological sample. In some embodiments, the level of the RNA is highly abundant in the biological sample as compared to the level of other RNA molecules in the biological sample.

In some embodiments, an analyte or target nucleic acid is RNA. In some embodiments, examples of RNA molecule include, without limitation, ribosomal RNA (rRNA), mitochondrial (mtRNA), messenger RNA (mRNA), transfer RNA (tRNA), and microRNA.

In some embodiments, an analyte or target nucleic acid is a ribosomal RNA (rRNA). Ribosomal RNA refers to ribosomal nucleic acid, which is the RNA component of ribosomes. Ribosomal RNA are transcribed in the nucleus, at specific structures called nucleoli. Nucleoli are dense, spherical shapes that form around genetic loci coding for rRNA. Non-limiting examples of rRNA include: 18S, 28S, 5.8S, 5S, 12S and 16S. In some embodiments, rRNA includes cytoplasmic rRNA, including: 18S, 28S, 5S, and 5.8S. In some embodiments, rRNA includes mitochondrial 12S and 16S. In some embodiments, IRNA include components of the large subunit of rRNA. In some embodiments, rRNA includes components of the small subunit of rRNA. In some embodiments, rRNA is encoding by the 45S transcription unit (e.g., 28S, 5.8S, 5S, and 18S rRNA). In some embodiments, rRNA is 18S rRNA. In some embodiments, rRNA is 28S rRNA. In some embodiments, rRNA is 5.8S rRNA. In some embodiments, rRNA is 12S rRNA. In some embodiments, rRNA is 16S rRNA. In some embodiments, rRNA is bacterial rRNA (e.g., 16S rRNA or 23S rRNA).

As used herein, “18S” refers to the 18S rRNA that is encoded by the 45S transcriptional subunit. A non-limiting example of a nucleic acid sequence encoding the 18S rRNA is NCBI reference sequence: NR_003286.4.

As used herein, “28S” refers to the 28S rRNA that is encoded by the 45S transcriptional subunit. A non-limiting example of a nucleic acid sequence encoding the 28s rRNA is NCBI reference sequence: NR_003287.4.

As used herein, “5.8S” refers to the 5.8S rRNA that is encoded by the 45S transcriptional subunit. A non-limiting example of a nucleic acid sequence encoding the 5.8s rRNA is NCBI reference sequence: NR_003285.3.

In some embodiments, the RNA molecule is mitochondrial RNA. Mitochondrial RNAs include, for example, 12S IRNA (encoded by MT-RNR1), and 16S rRNA (encoded by MT-RNR2), RNAs encoding electron transport chain proteins (e.g., NADH dehydrogenase, coenzyme Q-cytochrome c reductase/cytochrome b, cytochrome c oxidase, ATP synthase, or humanin), and tRNAs (encoded by MT-TA, MT-TR, MT-TN, MT-TD, MT-TC, MT-TE, MT-TQ, MT-TG, MT-TH, MT-TI, MT-TL1, MT-TL2, MT-TK, MT-TM, MT-TF, MT-TP, MT-TS1, MT-TS2, MT-TT, MT-TW, MT-TY, or MT-TV).

In some embodiments, the RNA is transfer RNA (tRNA). In some embodiments, the RNA may be a particular mRNA. For example, but is not limited to, ACTB, GAPDH, and TUBB. Other sequences for tRNA and specific mRNA are well known to those skilled in the art and can be readily found in sequence databases such as GenBank or may be found in the literature.

In some embodiments, the subset of nucleic acids includes mRNAs that mediate expression of a set of genes of interest. In some embodiments, the subset of nucleic acids includes mRNAs that share identical or substantially similar sequences, which mRNAs are translated into polypeptides having similar functional groups or protein domains. In some embodiments, the subset of nucleic acids includes mRNAs that do not share identical or substantially similar sequences, which mRNAs are translated into proteins that do not share similar functional groups or protein domains. In some embodiments, the subset of nucleic acids includes mRNAs that are translated into proteins that function in the same or similar biological pathways. In some embodiments, the biological pathways are associated with a pathologic disease. For example, targeted RNA capture can detect genes that are overexpressed or underexpressed in cancer.

In some embodiments, the subset of nucleic acids includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 target nucleic acids.

In some instances, the methods disclosed herein can detect the abundance and location of at least 5,000, 10,000, 15,000, 20,000, or more different nucleic acids.

In some instances, the probes are DNA probes. In some instances, the probes are diribo-containing probes.

Additional embodiments of probe(s) and probe set(s) are described herein.

### (i) First Probe

In some embodiments, the methods described herein include a first probe. As used herein, a “first probe” can refer to a probe that hybridizes to all or a portion of a nucleic acid (e.g., a ribosomal RNA) and can be ligated to one or more additional probes (e.g., a second probe). In some embodiments, “first probe” can be used interchangeably with “first probe oligonucleotide.”

In some embodiments, the first probe includes ribonucleotides, deoxyribonucleotides, and/or synthetic nucleotides that are capable of participating in Watson-Crick type or analogous base pair interactions. In some embodiments, the first probe includes deoxyribonucleotides. In some embodiments, the first probe includes deoxyribonucleotides and ribonucleotides. In some embodiments, the first probe includes a deoxyribonucleic acid that hybridizes to a nucleic acid, and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first probe includes deoxyribonucleotides, hybridization of the first probe to the mRNA molecule results in a DNA: RNA hybrid. In some embodiments, the first probe includes only deoxyribonucleotides and upon hybridization of the first probe to the mRNA molecule results in a DNA:RNA hybrid.

In some embodiments, the method includes a first probe that includes one or more sequences that are substantially complementary to one or more sequences of a target nucleic acid. In some embodiments, a first probe includes a sequence that is substantially complementary to a first target sequence in the target nucleic acid (e.g., an rRNA molecule). In some embodiments, the sequence of the first probe that is substantially complementary to the first target sequence in the target nucleic acid (e.g., a rRNA) is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the target nucleic acid (e.g., arRNA).

In some embodiments, a first probe includes a sequence at least 70%, at least 75%, at least 80%, at least 85%, at least 90%, at least 91%, at least 92%, at least 93%, at least 94%, at least 95%, at least 96%, at least 97%, at least 98%, or at least 99% identical to: 5′-CCTTGGCACCCGAGAATTCCATCCGTCTTGCGCCGGTCCAAGAATT-3′ (SEQ ID NO: 1). In some embodiments, a first probe includes a sequence that is at least 80% to SEQ ID NO: 1. In some embodiments, a first probe includes a sequence of SEQ ID NO: 1.

In some embodiments, a first probe includes a sequence that can hybridize to different nucleic acids. In some embodiments, a first probe includes a sequence that can hybridize to different nucleic acids from different subjects, wherein different subject means different species. For example, a first probe includes a sequence that can hybridize to a nucleic acid originating from a human biological sample and/or a nucleic acid originating from a mouse biological sample.

In some embodiments, a first probe includes a sequence that is about 10 nucleotides to about 100 nucleotides (e.g., a sequence of about 10 nucleotides to about 90 nucleotides, about nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a sequence of the first probe that is substantially complementary to a sequence in a nucleic acid includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some embodiments, a first probe includes a functional sequence. In some embodiments, a functional sequence includes a primer sequence.

In some embodiments, a first probe includes at least two ribonucleic acid bases at the 3′ end. In such cases, a second probe oligonucleotide includes a phosphorylated nucleotide at the 5′ end. In some embodiments, a first probe includes at least three, at least four, at least five, at least six, at least seven, at least eight, at least nine, or at least ten ribonucleic acid bases at the 3′ end.

As shown in FIG. 6, a non-limiting example of a first probe 601, which can be referred to as an LHS probe, includes a functional sequence 602, a sequence 603 that is substantially complementary to a first target sequence in the target nucleic acid 607.

### (ii) Second Probe

In some embodiments, the methods described herein include a second probe. As used herein, a “second probe” can refer to a probe that hybridizes to all or a portion of a target nucleic acid and can be ligated to one or more additional probes (e.g., a first probe). In some embodiments, “second probe” can be used interchangeably with “second probe oligonucleotide.” One of skill in the art will appreciate that the order of the probes is arbitrary, and thus the contents of the first probe and/or second probe as disclosed herein are interchangeable.

In some embodiments, the second probe includes ribonucleotides, deoxyribonucleotides, and/or synthetic nucleotides that are capable of participating in Watson-Crick type or analogous base pair interactions. In some embodiments, the second probe includes deoxyribonucleotides. In some embodiments, the second probe includes deoxyribonucleotides and ribonucleotides. In some embodiments, the second probe includes a deoxyribonucleic acid that hybridizes to a target nucleic acid and includes a portion of the oligonucleotide that is not a deoxyribonucleic acid. For example, in some embodiments, the portion of the second probe that is not a deoxyribonucleic acid is a ribonucleic acid or any other non-deoxyribonucleic acid nucleic acid as described herein. In some embodiments where the second probe includes deoxyribonucleotides, hybridization of the second probe to the mRNA molecule results in a DNA:RNA hybrid. In some embodiments, the second probe includes only deoxyribonucleotides and upon hybridization of the first probe to the mRNA molecule results in a DNA:RNA hybrid.

In some embodiments, the method includes a second probe that includes one or more sequences that are substantially complementary to one or more sequences of a target nucleic acid. In some embodiments, a second probe includes a sequence that is substantially complementary to a second target sequence in the target nucleic acid. In some embodiments, the sequence of the second probe that is substantially complementary to the second target sequence in the target nucleic acid is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the target nucleic acid.

In some embodiments, a second probe includes a sequence at least 70%, at least 75%, at least 80%, at least 85%, at least 90%, at least 91%, at least 92%, at least 93%, at least 94%, at least 95%, at least 96%, at least 97%, at least 98%, or at least 99% identical to: 5′-TCACCTCTAGCGGCGCAATACGAATAAAAAAAAAAAAAAAAAAAAAAAAAAAA AA-3′ (SEQ ID NO: 2). In some embodiments, a second probe includes a sequence that is at least 80% to SEQ ID NO: 2. In some embodiments, a second probe includes a sequence of SEQ ID NO: 2. In some embodiments, the second probe includes a free phosphate group at the 5′ end.

In some embodiments, a second probe includes a sequence that is about 10 nucleotides to about 100 nucleotides (e.g., a sequence of about 10 nucleotides to about 90) nucleotides, about 10 nucleotides to about 80 nucleotides, about 10 nucleotides to about 70) nucleotides, about 10 nucleotides to about 60 nucleotides, about 10 nucleotides to about 50) nucleotides, about 10 nucleotides to about 40 nucleotides, about 10 nucleotides to about 30) nucleotides, about 10 nucleotides to about 20 nucleotides, about 20 nucleotides to about 100 nucleotides, about 20 nucleotides to about 90 nucleotides, about 20 nucleotides to about 80) nucleotides, about 20 nucleotides to about 70 nucleotides, about 20 nucleotides to about 60 nucleotides, about 20 nucleotides to about 50 nucleotides, about 20 nucleotides to about 40) nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60) nucleotides, about 30 nucleotides to about 50 nucleotides, about 30 nucleotides to about 40) nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50) nucleotides, about 50 nucleotides to about 100 nucleotides, about 50 nucleotides to about 90) nucleotides, about 50 nucleotides to about 80 nucleotides, about 50 nucleotides to about 70) nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80) nucleotides, about 80) nucleotides to about 100 nucleotides, about 80) nucleotides to about 90) nucleotides, or about 90 nucleotides to about 100 nucleotides).

In some embodiments, a sequence of the second probe that is substantially complementary to a sequence in the target nucleic acid includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10) nucleotides to about 45 nucleotides, about 10 nucleotides to about 40 nucleotides, about 10) nucleotides to about 35 nucleotides, about 10 nucleotides to about 30 nucleotides, about 10) nucleotides to about 25 nucleotides, about 10 nucleotides to about 20 nucleotides, about 10) nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20) nucleotides to about 50 nucleotides, about 20 nucleotides to about 45 nucleotides, about 20) nucleotides to about 40 nucleotides, about 20 nucleotides to about 35 nucleotides, about 20) nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30) nucleotides to about 45 nucleotides, about 30 nucleotides to about 40 nucleotides, about 30) nucleotides to about 35 nucleotides, about 35 nucleotides to about 50 nucleotides, about 35 nucleotides to about 45 nucleotides, about 35 nucleotides to about 40 nucleotides, about 40) nucleotides to about 50 nucleotides, about 40 nucleotides to about 45 nucleotides, or about 45 nucleotides to about 50 nucleotides).

In some embodiments, a second probe includes a capture probe capture domain sequence. As used herein, a “capture probe capture domain” is a sequence, domain, or moiety that can specifically hybridize to a capture domain of a capture probe on a substrate. In some embodiments, “capture probe capture domain” can be used interchangeably with “capture probe binding domain.” In some embodiments, a second probe includes a sequence from 5′ to 3″: a sequence that is substantially complementary to a sequence in the target nucleic acid and a capture probe capture domain.

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain in a capture probe that detects a particular target(s) of interest. In some embodiments, a capture probe capture domain blocking moiety that interacts with the capture probe capture domain is provided. In some embodiments, a capture probe capture domain blocking moiety includes a sequence that is complementary or substantially complementary to a capture probe capture domain. In some embodiments, a capture probe capture domain blocking moiety prevents the capture probe capture domain from binding the capture probe when present. In some embodiments, a capture probe capture domain blocking moiety is removed prior to binding the capture probe capture domain (e.g., present in a ligated probe) to a capture probe. In some embodiments, a capture probe capture domain blocking moiety includes a poly-uridine sequence, a poly-thymidine sequence, or both. In some embodiments, the capture probe capture domain sequence includes ribonucleotides, deoxyribonucleotides, and/or synthetic nucleotides that are capable of participating in Watson-Crick type or analogous base pair interactions. In some embodiments, the capture probe binding domain sequence includes at least 10, 11, 12, 13, 14, 15, 16, 17, 18, 19, or 20 nucleotides. In some embodiments, the capture probe binding domain sequence includes at least 25, 30, or 35 nucleotides.

In some embodiments, a second probe includes a phosphorylated nucleotide at the 5′ end. The phosphorylated nucleotide at the 5′ end can be used in a ligation reaction to ligate the second probe to the first probe.

As shown in FIG. 6, a non-limiting example of a second probe 604, which can be referred to a RHS probe, includes a sequence 605 that is substantially complementary to a second target sequence on the target nucleic acid 607 and a capture probe capture domain 606.

In some embodiments, a second probe includes a sequence that can hybridize to different target nucleic acids. In some embodiments, a second probe includes a sequence that can hybridize to different nucleic acids from different subjects, wherein different subject means different species. For example, a second probe includes a sequence that can hybridize a nucleic acid originating from a human biological sample and a nucleic acid originating from a mouse biological sample.

(iii) Concentrations of First and Second Probes

In some embodiments, the method includes contacting the biological sample with a first probe and a second probe at a total concentration of about 25 nM to about 2500 nM (e.g., about 25 nM to about 2000 nM, about 25 nM to about 1500 nM, about 25 nM to about 1000 nM, about 25 nM to about 750 nM, about 25 nM to about 500 nM, about 25 nM to about 250 nM, about 25 nM to about 125 nM, about 25 nM to about 62.5 nM, about 25 nM to about 31.25 nM, about, about 31.25 nM to about 2500 nM, about 31.25 nM to about 2000 nM, about 31.25 nM to about 1500 nM, about 31.25 nM to about 1000 nM, about 31.25 nM to about 750 nM, about 31.25 nM to about 500 nM, about 31.25 nM to about 250 nM, about 31.25 nM to about 125 nM, about 31.25 nM to about 62.5 nM, about 62.5 nM to about 2500 nM, about 62.5 nM to about 2000 nM, about 62.5 nM to about 1500 nM, about 62.5 nM to about 1000 nM, about 62.5 nM to about 750 nM, about 62.5 nM to about 500 nM, about 62.5 nM to about 250 nM, about 62.5 nM to about 125 nM, about 125 nM to about 2500 nM, about 125 nM to about 2000 nM, about 125 nM to about 1500 nM, about 125 nM to about 1000 nM, about 125 nM to about 750 nM, about 125 nM to about 500 nM, about 125 nM to about 250 nM, about 250 nM to about 2500 nM, about 250 nM to about 2000 nM, about 250 nM to about 1500 nM, about 250 nM to about 1000 nM, about 250 nM to about 750 nM, about 250 nM to about 500 nM, about 500 nM to about 2500 nM, about 500 nM to about 2000 nM, about 500 nM to about 1500 nM, about 500 nM to about 1000 nM, about 500 nM to about 750 nM, about 750 nM to about 2500 nM, about 750 nM to about 2000 nM, about 750 nM to about 1500 nM, about 750 nM to about 1000 nM, about 1000 nM to about 2500 nM, about 1000 nM to about 2000 nM, about 1000 nM to about 1500 nM, about 1500 nM to about 2500 nM, about 2000 nM to about 2500 nM, or about 2000 nM to about 2500 nM). In some embodiments, the method includes contacting the biological sample the first probe and the second probe at a total concentration of about 2000 nM.

### (iv) Multiple Probes

In some embodiments, the methods disclosed herein include multiple probes. In some embodiments, the methods include 2, 3, 4, or more probes. In some embodiments, each of the probes includes ribonucleotides, deoxyribonucleotides, and/or synthetic nucleotides that are capable of participating in Watson-Crick type or analogous base pair interactions. In some embodiments, each of the probe includes deoxyribonucleotides. In some embodiments, each of the probe includes deoxyribonucleotides and ribonucleotides.

In some instances, the multiple probes span different target sequences, and multiple, serial ligation steps are carried out.

In some instances, the methods include a first probe and multiple second probes (or vice versa) are used, with the multiple second probes hybridizing to different sequences (e.g., wild-type versus mutant sequence, different isoforms, splice variants) in order to identify the sequence of a nucleic acid. It is appreciated that this method can be utilized to detect single mutations (e.g., point mutations, SNPs, splice variants, etc.) or can multi-nucleotide mutations (e.g., insertions, deletions, etc.).

Methods provided herein may be applied to a single nucleic acid molecule or a plurality of nucleic acid molecules. A method of analyzing a sample including a nucleic acid molecule may include providing a plurality of nucleic acid molecules (e.g., RNA molecules), where each nucleic acid molecule includes a first target region (e.g., a first target sequence) and a second target region (e.g., a second target sequence), a plurality of first probe oligonucleotides, and a plurality of second probe oligonucleotides. In some cases, one or more target regions of nucleic acid molecules of the plurality of nucleic acid molecules may include the same sequence. The first and second target regions (e.g., the first and second target sequences) of a nucleic acid molecule of the plurality of nucleic acid molecules may be adjacent to one another.

In some embodiments, the method also includes contacting the biological sample with one or more additional probe pairs, wherein a probe pair includes an additional first probe and an additional second probe. In some embodiments, each of the one or more additional probe pairs includes (i) a first sequence that is substantially complementary to a sequence of the rRNA, and (ii) a second sequence that is substantially complementary to a sequence of the rRNA, wherein the first sequence and the second sequence are adjacent in the IRNA. In some embodiments, each of the one or more additional probe pairs includes (i) a first sequence that is substantially complementary to a sequence of a second rRNA, and (ii) a second sequence that is substantially complementary to a sequence of a second rRNA, wherein the first sequence and the second sequence are adjacent in the second rRNA. In some embodiments, each of the one or more additional probe pairs includes (i) a first sequence that is substantially complementary to a sequence of a second target nucleic acid, and (ii) a second sequence that is substantially complementary to a sequence of a second target nucleic acid.

(c) Incorporation and Detection of Labelled dNTPs

In some embodiments, the methods described herein for testing a biological sample for efficacy of detection of a target nucleic acid includes a nucleic acid extension reaction where the ligation product hybridized to the capture probe is used as a template for extending the capture probe, thereby generating a sequence that is complementary to the ligation product. In such cases, incorporation of one or more labelled nucleotides (e.g., a nucleotide labelled with a fluorophore) enables detection of the sequence that is complementary to the ligation product, therein enabling detection of the ligation product itself.

In some embodiments, labelled nucleotides are incorporated enzymatically into DNA and RNA sequences for detection and analysis. In some embodiments, labeled nucleotides may be incorporated by a variety of methods including 3′ end labeling with terminal deoxynucleotidyl transferase (TdT), T4 DNA polymerase or T7 DNA polymerase, cDNA labeling with AMV or M-MuLV reverse transcriptase, and PCR labeling with thermophilic DNA polymerases like Taq, Pfu, Kapa HiFi DNA polymerase, and the like.

In some embodiments, labelled nucleotides are incorporated into a nucleic acid sequence using a reverse transcriptase buffer (Tris-HCl (pH 8.3), KCl, and MgCl2) with DTT and a DNA polymerase enzyme. For example, labelled nucleotides are incorporated into DNA sequences using an RT buffer (M-MLV Reverse Transcriptase Buffer is supplied as 5×Buffer (Tris-HCl (pH 8.3), KCl, MgCl2) with 5DTT and a Kapa HiFi DNA polymerase.

In some embodiments, labelled nucleotides are incorporated into a nucleic acid sequence using a second strand synthesis buffer and a DNA polymerase enzyme. In some embodiments, the second strand synthesis buffer includes glycerol. In some embodiments, the second strand synthesis buffer includes tris-HCl. In some embodiments, the second strand synthesis buffer includes KCl. In some embodiments, the second strand synthesis buffer includes MgCl₂. In some embodiments, the second strand synthesis buffer includes a surfactant (e.g., synperonic F108). In some embodiments, the second strand synthesis buffer includes biased dNTP and dCTP-Cy3. In some embodiments, the second strand synthesis buffer includes a stabilization agent (e.g., bovine serum albumin). In some embodiments, the second strand synthesis buffer includes glycerol, tris-HCl, KCl, MgCl₂, a surfactant (e.g., synperonic F108), biased dNTP and dCTP-Cy3, and a stabilization agent (e.g., bovine serum albumin).

In some embodiments, labelled nucleotides are incorporated into a nucleic acid sequence using a reverse transcriptase enzyme. Non-limiting examples of reverse transcriptases that can be used to incorporate labelled nucleotides into a nucleic acid sequence include: avian myeloblastosis virus (AMV) reverse transcriptase, Moloney murine leukemia virus (M-MuLV or MMLV) reverse transcriptase, HIV reverse transcriptase, ArrayScript™, MultiScribe™, ThermoScript™, and SuperScript® I, II, III, and IV reverse transcriptases.

In some embodiments, labelled nucleotides are incorporated into a nucleic acid sequence using a DNA polymerase. Non-limiting examples of DNA polymerases that can be used to incorporate labelled nucleotides into a nucleic acid sequence include DNA-dependent DNA polymerases (e.g., phi29 DNA polymerase and Taq DNA polymerase).

In some embodiments, a nucleotide is labelled with a detectable label. In some embodiments, a detectable label can include a radiolabel, a fluorescent label, a chemiluminescent label, a bioluminescent label, a calorimetric label, or a colorimetric label, or combinations thereof.

In some embodiments, the detectable label is a fluorophore. For example, the fluorophore can be from a group that includes: 7-AAD (7-Aminoactinomycin D). Acridine Orange (+DNA), Acridine Orange (+RNA), Alexa Fluor® 350. Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633 Alexa Fluor® 647, Alexa Fluor® 660 Alexa Fluor® 680, Alexa Fluor® 700, Alexa Fluor® 750, Allophycocyanin (APC), AMCA/AMCA-X, 7-Amino-4-methylcoumarin. 6-Aminoquinoline. Aniline Blue, ANS, APC-Cy7. ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, CF dyes, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DID (DilC18(5)), DIDS, Dil (DilC18(3)), DiO (DiOC18(3)), DIR (DilC18(7)), Di-4 ANEPPS, Di-8 ANEPPS, DM-NERF (4.5-6.5 pH), DsRed (Red Fluorescent Protein), EBFP, ECFP, EGFP, ELFR;-97 alcohol, Eosin, Erythrosin, Ethidium bromide, Ethidium homodimer-1 (EthD-1), Europium (III) Chloride, 5-FAM (5-Carboxyfluorescein), Fast Blue. Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1.5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, Mito Tracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66 W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550), ATTO 565, ATTO Rho101, ATTO 590, ATTO 633, ATTO 647N, TYE 563, TYE 615, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, a detectable label is a radiolabel. In some embodiments, the radiolabel is a beta-emitter. In some embodiments, the labelled nucleotides include ³²p, ³³p and ³⁵S labelled nucleotides. In some embodiments, the radiolabel (e.g., ³²p, ³³P and ³⁵S) is present on the alpha phosphate group or the gamma phosphate group (i.e., the phosphate groups of the nucleoside triphosphate). Others have demonstrated methods using radiolabeled nucleotides. See, e.g., Berent et al., Bio-Techniques 3: 208-220 (1985) and Friedman et al., Nucleic Acids Res., 4, 3455-3471 (1977), both of which are herein incorporated by reference in their entirety.

In some embodiments, generating a sequence that is complementary to the hybridized ligation product and detecting a signal corresponding to the ligation product on the substrate includes incorporating affinity group labeled dNTPs into the sequence. In such cases, the sequence that is complementary to the ligation product that is hybridized to a capture probe can be detected with affinity group conjugates. For example, generating a sequence that is complementary to the hybridized ligation product includes incorporation of biotin labelled dNTPs (e.g., aha-dUTP and/or aha-dCTP) that can be detected with avidin conjugates (e.g., avidin, streptavidin, neutravidin, and captavidin). In another example, generating a sequence that is complementary to the hybridized ligation product includes incorporation of a fluorescein-labeled dNTP (e.g., aha-dUTP) that can be detected with labeled anti-fluorescein antibodies. In some embodiments, the signal from biotin-labeled nucleotides incorporated into the sequence that is complementary to the ligation product can be amplified. Non-limiting examples of signal amplification include tyramide signal amplification technology (TSA) and peroxidase-based signal amplification techniques.

In some embodiments, the method includes detecting a signal corresponding to the ligation product on the substrate, thereby determining the efficacy of detection of the target nucleic acid. In some embodiments, the signal corresponding to the hybridized ligation product includes the signal from the labelled dNTPs incorporated into the sequence complementary to the ligation product hybridized to the capture probe.

In some embodiments, the method includes detecting a signal, and correlating the intensity of the signal with the efficiency of incorporation of the one or more labelled nucleotides into the generated sequence thereby determining the reaction condition for efficiently detecting the target nucleic acid in the biological sample. In some instances, the methods include correlating the intensity of the signal further determines degradation status of the target nucleic acid.

Degradation status can be a relative or an absolute value. When degradation status of a target nucleic acid is a relative value, the status (e.g., intensity of signal) is compared to status (e.g., intensity of signal) in a reference sample. A reference sample in some instances can be a sample of the same tissue, age, and under similar storage conditions. In other instances, a reference sample can be a sample that has been prepared under more optimal conditions such as shorter storage time, less or no treatment with a fixative (e.g., such as a fresh frozen tissue). In other instances, the reference sample can be a sample which is known to provide a certain signal intensity under conditions of interest (e.g., under certain permeabilization or concentration conditions). When a degradation status is an absolute value, it can be determined using methods known in the art to assign an arbitrary value to the intensity. For instance, a signal intensity can be assigned an absolute value based on pixel intensity of the signal using a fluorescent microscope. One skilled in the art then can determine a threshold value or percent degradation of a target nucleic acid based on the intensity of the signal (either relative or absolute). For example, a reference sample can be designated as having a threshold signal for nucleic acids in a sample to be called “not degraded” and thus any biological sample having a signal intensity at or above this threshold (e.g., under certain conditions) would also include nucleic acids that could be considered “not degraded” or of sufficient quality for additional similar experiments. On the other hand, if a sample were to have a signal intensify lower than the threshold, then one could consider the nucleic acids form that sample “degraded” or of insufficient quality for additional similar experiments.

In some embodiments, the detecting step includes obtaining an image corresponding to the signal corresponding to the ligation product that is hybridized to a capture probe on the substrate. In some embodiments, detecting the signal corresponding to the hybridized ligation product includes obtaining an image corresponding to the labelled dNTPs incorporated into the sequence complementary to the ligation product hybridized to the capture probe. In some embodiments, the method also includes registering image coordinates to a fiducial marker. As used herein, fiducial marker refers to objects placed in the field of view of an imaging system which appear in the image produced. Fiducial markers can be used as a point of reference or measurement scale for alignment to determine a location of a sample or array on a substrate relative to a fiducial marker and/or for quantitative measurements of sizes and/or distances.

In some embodiments, the method also includes analyzing a signal corresponding to the ligation product that is hybridized to the capture probe on the substrate. In some embodiments, analysis of a signal corresponding to the ligation product hybridized to the capture probe includes analysis of the signal from the labelled dNTPs.

In some embodiments, the method includes identifying, based on the signal analysis, a biological sample wherein the target nucleic acids can be used for detection of a target nucleic acid using targeted RNA capture. In some embodiments, the method includes identifying, based on the signal analysis, the permeabilization conditions for a biological sample. As such, the methods herein can determine when the nucleic acids in a biological sample is of sufficient quality to continue with the sample for spatial gene expression array analysis, and the permeabilization conditions that can be used for target nucleic acid capture from that biological sample.

### (d) Pre-Hybridization Methods

### (i) Imaging and Staining

Prior to addition of the probes, in some instances, biological samples can be stained using a wide variety of stains and staining techniques. In some instances, the biological sample is a tissue section (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying target nucleic acids using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017: 65(8): 431-444, Lin et al., Nat Commun. 2015: 6:8390, Pirici et al., J. Histochem. Cytochem. 2009: 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped, imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization is useful for identifying intracellular nucleic acids. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of a Sample for Application of Probes

In some instances, the biological sample is deparaffinized, for example if using a formalin fixed paraffin embedded biological sample. Deparaffinization can be achieved using any method known in the art. For example, in some instances, the biological sample is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each: 95% ethanol, two washes 10 minutes each: 70% ethanol, two washes 10 minutes each: 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked in order to relieve nucleic acids that were crosslinked to proteins during the formalin fixation step. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, the methods of preparing a biological sample for probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each: 3 times at 5 minutes each). In some instances, the biological sample is blocked with a blocking buffer. In some instances, the blocking buffer includes a carrier such as RNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (e) Hybridizing the Probes

In some embodiments, the methods of targeted RNA capture provided herein include hybridizing a first probe oligonucleotide and a second probe oligonucleotide (e.g., a probe pair). In some instances, the first and second probe oligonucleotides each include sequences that are substantially complementary to one or more sequences (e.g., one or more target sequences) of a nucleic acid of interest. In some embodiments, the first probe and the second probe bind to complementary sequences that are completely adjacent (i.e., no gap of nucleotides) to one another or are on the same nucleic acid.

In some instances, the methods include hybridization of probe sets, wherein the probe pairs are in a medium at a concentration of about 1 to about 100 nM. In some instances, the concentration of the probe pairs is about 1, 2, 3, 4, 5, 6, 7, 8, 9, 10, 15, 20, 25, 30, 35, 40, 45, 50, 60, 70, 80, 90, 100, 200, 300, 400, or 500 nM. In some instances, the concentration of the probe pairs is 5 nM. In some instances, the probe sets are diluted in a hybridization (Hyb) buffer. In some instances, the probe sets are at a concentration of 5 nM in Hyb buffer.

In some instances, probe hybridization occurs at about 50° C. In some instances, the temperature of probe hybridization ranges from about 30° C. to about 75° C., from about 35° C. to about 70° C., or from about 40° C. to about 65° C. In some instances, probe hybridization occurs for about 30 minutes, about 1 hour, about 2 hours, about 2.5 hours, about 3 hours, or more. In some instances, probe hybridization occurs for about 2.5 hours at 50° C.

In some instances, the hybridization buffer includes SSC (e.g., 1×SSC) or SSPE. In some instances, the hybridization buffer includes formamide or ethylene carbonate. In some instances, the hybridization buffer includes one or more salts, like Mg salt for example MgCl₂, Na salt for example NaCl, Mn salt for example MnCl₂. In some instances, the hybridization buffer includes Denhardt's solution, dextran sulfate, ficoll, PEG or other hybridization rate accelerators. In some instances, the hybridization buffer includes a carrier such as yeast tRNA, salmon sperm DNA, and/or lambda phage DNA. In some instances, the hybridization buffer includes one or more blockers. In some instances, the hybridization buffer includes RNase inhibitor(s). In some instances, the hybridization buffer can include BSA, sequence specific blockers, non-specific blockers, EDTA, RNase inhibitor(s), betaine, TMAC, or DMSO. In some instances, a hybridization buffer can further include detergents such as Tween, Triton-X 100, sarkosyl, and SDS. In some instances, the hybridization buffer includes nuclease-free water, DEPC water.

In some embodiments, the complementary sequences to which the first probe oligonucleotide and the second probe oligonucleotide bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other. Gaps between the probe oligonucleotides may first be filled prior to ligation, using, for example, DNA polymerase, RNA polymerase, reverse transcriptase, VENT polymerase, Taq polymerase, and/or any combinations, derivatives, and variants (e.g., engineered mutants) thereof. In some embodiments, when the first and second probe oligonucleotides are separated from each other by one or more nucleotides, nucleotides are ligated between the first and second probe oligonucleotides. In some embodiments, when the first and second probe oligonucleotides are separated from each other by one or more nucleotides, deoxyribonucleotides are ligated between the first and second probe oligonucleotides.

In some instances, after hybridization, the biological sample is washed with a post-hybridization wash buffer. In some instances, the post-hybridization wash buffer includes one or more of SSC, yeast tRNA, formamide, ethylene carbonate, and nuclease-free water.

In some embodiments, a ligation step is performed. Ligation can be performed using any of the methods described herein. In some embodiments, the step includes ligation of the first oligonucleotide (e.g., the first probe) and the second oligonucleotide (e.g., the second probe), forming a ligation product. In some embodiments, ligation is chemical ligation. In some embodiments, ligation is enzymatic ligation. In some embodiments, the ligase is a T4 RNA ligase (Rnl2), a PBCV-1, a ligase from a Chlorella virus, a ligase that will ligate two single stranded DNA molecules that are adjacently positioned on an RNA sequence, a single stranded DNA ligase, or a T4 DNA ligase.

### (i) Hybridization Buffer

In some embodiments, a first probe and a second probe are hybridized to a target nucleic acid in a hybridization buffer. In some instances, the hybridization buffer contains formamide. In other instances, the hybridization buffer is formamide free. Formamide is not human friendly and it is a known health hazard. Chemically, it can oxidize over time, thereby impacting reagent shelf life and, most importantly, reagent efficacy. As such, the methods described herein can include formamide-free buffers, including formamide-free hybridization buffer.

In some embodiments, the formamide-free hybridization buffer is a saline-sodium citrate (SSC) hybridization buffer. In some embodiment, the SSC is present in the SSC hybridization buffer from about 1×SSC to about 6×SSC (e.g., about 1×SSC to about 5×SSC, about 1×SSC to about 4×SSC, about 1×SSC to about 3×SSC, about 1×SSC to about 2×SSC, about 2×SSC to about 6×SSC, about 2×SSC to about 5×SSC, about 2×SSC to about 4×SSC, about 2×SSC to about 3×SSC, about 3×SSC to about 5×SSC, about 3×SSC to about 4×SSC, about 4×SSC to about 6×SSC, about 4×SSC to about 6×SSC, about 4×SSC to about 5×SSC, or about 5×SSC to about 6×SSC). In some embodiments, the SSC is present in the SSC hybridization buffer from about 2×SSC to about 4×SSC. In some embodiments, SSPE hybridization buffer can be used.

In some embodiments, the SSC hybridization buffer comprises a solvent. In some embodiments, the solvent comprises ethylene carbonate instead of formamide (2020, Kalinka et al., Scientia Agricola 78(4): e20190315). In some embodiments, ethylene carbonate is present in the SSC hybridization buffer from about 10% (w/v) to about 25% (w/v) (e.g., about 10% (w/v) to about 20% (w/v), about 10% (w/v) to about 15% (w/v), about 15% (w/v) to about 25% (w/v), about 15% (w/v) to about 20% (w/v), or about 20% (w/v) to about 25% (w/v)). In some embodiments, ethylene carbonate is present in the SSC hybridization buffer from about 15% (w/v) to about 20% (w/v). In some embodiments, ethylene carbonate is present in the SSC hybridization buffer at about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), about 20% (w/v), about 21% (w/v), about 22% (w/v), about 23% (w/v), about 24% (w/v), or about 25% (w/v). In some embodiments, ethylene carbonate is present in the SSC hybridization buffer at about 13% (w/v).

In some embodiments, the SSC hybridization buffer is at a temperature from about 40° C. to about 60° C. (e.g., about 40° C. to about 55° C., about 40° C. to about 50° C., about 40° C. to about 45° C., about 45° C. to about 60° C., about 45° C. to about 55° C., about 45° C. to about 50° C., about 50° C. to about 60° C., about 50° C. to about 55° C., or about 55° C. to about 60° C.). In some embodiments, the SSC hybridization buffer is at temperature from about 45° C. to about 55° C., or any of the subranges described herein. In some embodiments, the SSC hybridization buffer is at a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some embodiments, the SSC hybridization buffer is at a temperature of about 50° C.

In some embodiments, the SSC hybridization buffer further comprises one or more of a carrier, a crowder, or an additive. Non-limiting examples of a carrier that can be included in the hybridization buffer include: yeast tRNA, salmon sperm DNA, lambda phage DNA, glycogen, and cholesterol. Non-limiting examples of a molecular crowder that can be included in the hybridization buffer include: Ficoll, dextran, Denhardt's solution, and PEG. Non-limiting examples of additives that can be included in the hybridization buffer include:

binding blockers, RNase inhibitors, Tm adjustors and adjuvants for relaxing secondary nucleic acid structures (e.g., betaine, TMAC, and DMSO). Further, a hybridization buffer can include detergents such as SDS, Tween, Triton-X 100, and sarkosyl (e.g., N-LauroyIsarcosine sodium salt). A skilled artisan would understand that a buffer for hybridization of nucleic acids could include many different compounds that could enhance the hybridization reaction.

(f) Washing In some embodiments, the methods disclosed herein also include a wash step. The wash step removes any unbound probes. Wash steps could be performed between any of the steps in the methods disclosed herein. For example, a wash step can be performed after adding probes to the biological sample. As such, free/unbound probes are washed away, leaving only probes that have hybridized to a target nucleic acid. In some instances, multiple (i.e., at least 2, 3, 4, 5, or more) wash steps occur between the methods disclosed herein. Wash steps can be performed at times (e.g., 1, 2, 3, 4, or 5 minutes) and temperatures (e.g., room temperature: 4° C. known in the art and determined by a person of skill in the art.

In some instances, wash steps are performed using a wash buffer. In some instances, the wash buffer includes SSC (e.g., 1×SSC). In some instances, the wash buffer includes PBS (e.g., 1×PBS). In some instances, the wash buffer includes PBST (e.g., 1×PBST). In some instances, the wash buffer can also include formamide or be formamide free.

In some embodiments, after ligating a first probe and a second probe, the one or more unhybridized first probes, one or more unhybridized second probes, or both, are removed from the array. In some embodiments, after ligating a first probe and a second probe, the one or more unhybridized first and/or second are removed from the array. In some embodiments, after ligating a first probe, a second probe, and a third oligonucleotide, the one or more unhybridized first probes, one or more unhybridized second probes, or one or more third oligonucleotides, or all the above, are removed from the array.

In some embodiments, a pre-hybridization buffer is used to wash the sample. In some embodiments, a phosphate buffer is used. In some embodiments, multiple wash steps are performed to remove unbound oligonucleotides.

In some embodiments, the SSC wash buffer comprises a detergent. In some embodiments, the detergent comprises sodium dodecyl sulfate (SDS). In some embodiments, SDS is present in the SSC wash buffer from about 0.01% (v/v) to about 0.5% (v/v). In some embodiments, the SDS is present in the SSC wash buffer at about 0.1% (v/v). In some embodiments, sarkosyl may be present in the SSC wash buffer.

In some embodiments, the SSC wash buffer comprises a solvent. In some embodiments, the solvent comprises ethylene carbonate. In some embodiments, ethylene carbonate is present in the SSC wash buffer from about 10% (w/v) to about 25% (w/v), or any of the subranges described herein. In some embodiments, ethylene carbonate is present in the SSC wash buffer from about 15% (w/v) to about 20% (w/v). In some embodiments, ethylene carbonate is present in the SSC wash buffer at about 16% (w/v).

In some embodiments, the SSC wash buffer is at a temperature from about 50° C. to about 70° C. (e.g., about 50° C. to about 65° C., about 50° C. to about 60° C., about 50° C. to about 55° C., about 55° C. to about 70° C., about 55° C. to about 65° C., about 55° C. to about 60° C., about 60° C. to about 70° C., about 60° C. to about 65° C., or about 65° C. to about 70° C.). In some embodiments, the SSC wash buffer is at a temperature from about 55° C. to about 65° C. In some embodiments, the SSC wash buffer is at a temperature of about 60° C.

In some embodiments, the method includes releasing the ligation product, where releasing is performed after the array is washed to remove the one or more unhybridized first and second probes.

### (g) Ligation

In some embodiments, after hybridization of the probes (e.g., a first probe and a second probe) to the target nucleic acid, the probes (e.g., a first probe or a second probe) can be ligated together, creating a single ligation product that includes one or more sequences that are complementary to the nucleic acid. Ligation can be performed enzymatically or chemically, as described herein.

In some instances, the ligation is an enzymatic ligation reaction, using a ligase (e.g., T4 RNA ligase (Rnl2), a PBCV-1 ligase, a ligase from a Chlorella virus, a ligase that will ligate two DNA strands that are adjacently positioned on a RNA sequence, a single stranded DNA ligase, or a T4 DNA ligase). See, e.g., Zhang et al.; RNA Biol. 2017: 14(1): 36-44, which is incorporated by reference in its entirety, for a description of KOD ligase. Following the enzymatic ligation reaction, the probes (e.g., a first probe and a second probe) may be considered ligated.

In some embodiments, a polymerase catalyzes synthesis of a complementary strand of the ligation product, creating a double-stranded ligation product. In some instances, the polymerase is DNA polymerase. In some embodiments, the polymerase has 5′ to 3″ polymerase activity. In some embodiments, the polymerase has 3′ to 5′ exonuclease activity for proofreading. In some embodiments, the polymerase has 5′ to 3′ polymerase activity and 3′ to 5′ exonuclease activity for proofreading.

In some embodiments, the probe (e.g., a first probe or a second probe) may each include a reactive moiety such that, upon hybridization to the target and exposure to appropriate ligation conditions, the probes may ligate to one another. In some embodiments, probes that include a reactive moiety are ligated chemically. For example, a first probe capable of hybridizing to a first target region (e.g., a first target sequence or a first portion) of a nucleic acid molecule may include a first reactive moiety, and a second probe oligonucleotide capable of hybridizing to a second target region (e.g., a second target sequence or a second portion) of the nucleic acid molecule may comprise a second reactive moiety. When the first and second probes are hybridized to the first and second target regions (e.g., first and second target sequences) of the nucleic acid molecule, the first and second reactive moieties may be adjacent to one another. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phorphorothioate), acids, amines, and phosphates. For example, the first reactive moiety of a first probe may comprise an azide moiety, and a second reactive moiety of a second probe may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition: a thiol-ene reaction: a nucleophilic substation reaction: or another reaction. In some cases, reaction between the first and second reactive moieties may yield a triazole moiety or an isoxazoline moiety. A reaction between the first and second reactive moieties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe hybridized to a first target region (e.g., a first target sequence or first portion) of the nucleic acid molecule and a second reactive moiety of a third probe oligonucleotide hybridized to a second target region (e.g., a first target sequence or a first portion) of the nucleic acid molecule may link the first probe and the second probe to provide a ligated probe. Upon linking, the first and second probe may be considered ligated. Accordingly, reaction of the first and second reactive moieties may comprise a chemical ligation reaction such as a copper-catalyzed 5′ azide to 3′ alkyne “click” chemistry reaction to form a triazole linkage between two probe oligonucleotides. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond. In some instances, ligation is performed in a ligation buffer. In instances where probe ligation is performed on diribo-containing probes, the ligation buffer can include T4 RNA Ligase Buffer 2, enzyme (e.g., RNL2 ligase), and nuclease free water. In instances where probe ligation is performed on DNA probes, the ligation buffer can include Tris-HCl pH7.5, MnC12, ATP, DTT, surrogate fluid (e.g., glycerol), enzyme (e.g., Chlorella virus ligase, PBCV-1 ligase), and nuclease-free water.

In some embodiments, the ligation buffer includes additional reagents. In some instances, the ligation buffer includes adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers the AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g. because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 μM during the ligation reaction.

In some embodiments, cofactors that aid in joining of the probe oligonucleotides are added during the ligation process. In some instances, the cofactors include magnesium ions (Mg²⁺). In some instances, the cofactors include manganese ions (Mn²⁺). In some instances, Mg²⁺ is added in the form of MgCl₂. In some instances, Mn²⁺ is added in the form of MnCl₂. In some instances, the concentration of MgCl₂ is at about 1 mM, at about 10 mM, at about 100 mM, or at about 1000 mM. In some instances, the concentration of MnCl₂ is at about 1 mM, at about 10 mM, at about 100 mM, or at about 1000 mM.

In some instances, the ligation occurs at a pH in the range of about 6.5 to about 9.0, about 6.5 to about 8.0, or about 7.5 to about 8.0.

In some embodiments, the ligation buffer includes an enzyme storage buffer. In some embodiments, the enzymes storage buffer includes glycerol. In some embodiments, the ligation buffer is supplemented with glycerol. In some embodiments, the glycerol is present in the ligation buffer at a total volume of 15% v/v.

In some embodiments, the ligation product includes a capture probe capture domain, which can bind to a capture probe (e.g., a capture probe immobilized, directly or indirectly, on a substrate). In some embodiments, methods provided herein include contacting a biological sample with a substrate, wherein the capture probe is affixed to the substrate (e.g., immobilized to the substrate, directly or indirectly). In some embodiments, the capture probe capture domain of the ligated probe specifically binds to the capture domain.

After ligation, in some instances, the biological sample is washed with a post-ligation wash buffer. In some instances, the post-ligation wash buffer includes one or more of SSC (e.g., 1×SSC), ethylene carbonate or formamide, and nuclease free water. In some instances, the biological sample is washed at this stage at about 50° C. to about 70° C. In some instances, the biological sample is washed at about 60° C.

(h) Permeabilization and Releasing the Ligation Product In some embodiments, the methods provided herein include a permeabilizing step. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, after creating a ligation product (e.g., by ligating a first probe and a second probe that are hybridized to adjacent sequences in the target nucleic acid), the biological sample is permeabilized. In some embodiments, the biological sample is permeabilized contemporaneously with or prior to contacting the biological sample with a first probe and a second probe, hybridizing the first probe and the second probe to the target nucleic acid, generating a ligation product by ligating the first probe and the second probe, and releasing the ligated product from the target nucleic acid.

In some embodiments, methods provided herein include permeabilization of the biological sample such that the capture probe is more readily accessible for hybridizing to the captured ligated probe (i.e., compared to no permeabilization). In some embodiments, reverse transcription (RT) reagents can be added to permeabilized biological samples. Incubation with the RT reagents can produce sequences complementary to the ligation product that is hybridized to the capture probe. In some embodiments, second strand reagents (e.g., second strand primers, enzymes, labeled and unlabeled dNTPs) can be added to the biological sample on the slide to initiate second strand synthesis.

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K), and nuclease free water.

In some instances, the permeabilization buffer includes a ribonuclease inhibitor. In some instances, the ribonuclease inhibitor includes ribonucleoside vanadyl complex (RVC). In some instances, the permeabilization buffer includes a ribonuclease inhibitor and a reducing agent. For example, the permeabilization buffer includes RVC and DTT. In some instances, RVC is added to the permeabilization buffer at a final concentration of about 2 mM to about 20 mM (e.g., about 2 mM to about 15 mM, about 2 mM to about 10 mM, about 2 mM to about 5 mM, about 5 mM to about 20 mM, about 5 mM to about 15 mM, about 5 mM to about 10 mM, about 10 mM to about 20 mM, about 10 mM to about 15 mM, or about 15 mM to about 20 mM). In some instances, RVC is added to the permeabilization at a final concentration of about 10 mM.

In some instances, the permeabilization step is performed at 37° C. In some instances, the permeabilization step is performed for about 20 minutes to 2 hours (e.g., about 20) minutes, about 30 minutes, about 40 minutes, about 50 minutes, about 1 hour, about 1.5 hours, or about 2 hours). In some instances, the releasing step is performed for about 40) minutes. In some embodiments, the permeabilization parameters are varied such that the optimal permeabilization conditions for a particular tissue type or sample can be determined for optimal tissue nucleic acid capture by the capture probe on the substrate.

In some embodiments, after generating a ligation product, the ligation product is released from the target nucleic acid. In some embodiments, a ligation product is released from the nucleic acid using an endoribonuclease. In some embodiments, the endoribonuclease is RNase H, RNase A, RNase C, or RNase I. In some embodiment, the endoribonuclease is RNase A. In some embodiments, the endoribonuclease is RNase H. RNase H is an endoribonuclease that specifically hydrolyzes the phosphodiester bonds of RNA, when hybridized to DNA. RNase H is part of a conserved family of ribonucleases which are present in many different organisms. There are two primary classes of RNase H; RNase H1 and RNase H2. Retroviral RNase H enzymes are similar to the prokaryotic RNase H1. All of these enzymes share the characteristic that they are able to cleave the RNA component of an RNA:DNA heteroduplex. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli.

In some instances, the releasing step is performed using a releasing buffer. In some instances, the releasing buffer includes one or more of a buffer (e.g., Tris pH 7.5), enzyme (e.g., RNAse H) and nuclease-free water. In some instances, the releasing step is performed at 37° C. In some instances, the releasing step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 30 minutes.

In some instances, the releasing step occurs before the permeabilization step. In some instances, the releasing step occurs after the permeabilization step. In some instances, the releasing step occurs at the same time as the permeabilization step (e.g., in the same buffer).

### (i) Blocking Probes

In some embodiments, a capture probe capture domain is blocked prior to adding a second probe oligonucleotide to a biological sample. This prevents the capture probe capture domain from prematurely hybridizing to the capture domain.

In some embodiments, a blocking probe is used to block or modify the free 3′ end of the capture probe capture domain. In some embodiments, a blocking probe can be hybridized to the capture probe capture domain of the second probe to mask the free 3′ end of the capture probe capture domain. In some embodiments, a blocking probe can be a hairpin probe or partially double stranded probe. In some embodiments, the free 3′ end of the capture probe capture domain of the second probe can be blocked by chemical modification, e.g., addition of an azidomethyl group as a chemically reversible capping moiety such that the capture probes do not include a free 3′ end. Blocking or modifying the capture probe capture domain, particularly at the free 3′ end of the capture probe capture domain, prior to contacting second probe with the substrate, prevents hybridization of the second probe to the capture domain (e.g., prevents the capture of a poly(A) of a capture probe capture domain to a poly(T) capture domain). In some embodiments, a blocking probe can be referred to as a capture probe capture domain blocking moiety.

In some embodiments, the blocking probes can be reversibly removed. For example, blocking probes can be applied to block the free 3′ end of either or both the capture probe capture domain and/or the capture probes. Blocking interaction between the capture probe capture domain and the capture probe on the substrate can reduce non-specific capture to the capture probes. After the second probe hybridizes to the target nucleic acid and is ligated to a first probe, the blocking probes can be removed from the 3′ end of the capture probe capture domain and/or the capture probe, and the ligation product can migrate to and hybridize to the capture probes on the substrate. In some embodiments, the removal includes denaturing the blocking probe from capture probe capture domain and/or capture probe. In some embodiments, the removal includes removing a chemically reversible capping moiety. In some embodiments, the removal includes digesting the blocking probe with an RNase (e.g., RNase H).

In some embodiments, the blocking probes are oligo (dT) blocking probes. In some embodiments, the oligo (dT) blocking probes can have a length of 15-30 nucleotides. In some embodiments, the oligo (dT) blocking probes can have a length of 10-50 nucleotides, e.g., 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 nucleotides. In some embodiments, the analyte capture agents can be blocked at different temperatures (e.g., 4° C. and 37° C.).

### (j) Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells. In some instances, the biological sample is a cell pellet.

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and the nucleic acids are fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

### (k) Determining the Sequence of the Ligation Product

In some cases, testing a biological sample for the ability of the nucleic acids to be effectively detected (or determining permeabilization conditions) identifies a biological sample that has nucleic acids that are suitable for additional analysis, including targeted RNA capture. The method can also include selecting, based on the detected signal, a second tissue section of the biological sample for the detection of one or more additional nucleic acids. In such cases, the method also includes analyzing a second portion or tissue section of the biological sample for the detection of one or more additional target nucleic acids, wherein analyzing the second tissue section comprises determining a location and abundance of one or more nucleic acids in the second tissue section of the biological sample. In some embodiments, the second tissue section can be a serial section of the biological sample that was used to determine the state (e.g. degraded, not degraded, etc.) of the nucleic acids to be effectively detected. In some embodiments, the second tissue section can be a serial section of the biological sample that was used to determine permeabilization conditions for targeted RNA capture.

In some embodiments, the steps of analyzing a biological sample for one or more additional nucleic acids includes: (a) providing the second tissue section of the biological sample on a second array comprising a second plurality of second capture probes, wherein a second capture probe of the second plurality of second capture probes comprises: (i) a spatial barcode and (ii) a second capture domain; (b) applying a second plurality of probes on the biological sample, wherein a first probe of the second plurality of probes and a second probe of the second plurality of probes each comprise sequences that are substantially complementary to sequences of the nucleic acid, and wherein the second probe of the second plurality of probes comprises a second capture domain; (c) generating a second ligation product by ligating the first probe of the second plurality of probes and the second probe of the second plurality of probes; (d) releasing the second ligation product from the nucleic acid; (e) hybridizing the second ligation product to the second capture domain; and (f) determining (i) all or a part of the sequence of the second ligation product hybridized to the capture domain, or a complement thereof, and (ii) the sequence of the spatial barcode, or a complement thereof, and using the determined sequence of (i) and (ii) to identify the location of the nucleic acid in the biological sample.

In some embodiments, provided herein are methods for spatially detecting a target nucleic acid (e.g., detecting the location of the nucleic acid) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological nucleic acids; and (d) analyzing the captured biological nucleic acid, thereby spatially detecting the biological nucleic acid; wherein the biological sample is fully or partially removed from the substrate.

In some embodiments, a biological sample is not removed from the substrate. For example, the biological sample is not removed from the substrate prior to releasing a capture probe (e.g., a capture probe bound to a nucleic acid) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to a target nucleic acid can be made and the copy can be released from the substrate, e.g., via denaturation). In some embodiments, the biological sample is not removed from the substrate prior to analysis of the nucleic acid hybridized to a capture probe after it is released from the substrate. In some embodiments, the biological sample remains on the substrate during removal of a capture probe from the substrate and/or analysis of the nucleic acid hybrdized to the capture probe after it is released from the substrate. In some embodiments, the biological sample remains on the substrate during removal (e.g., via denaturation) of a copy of the capture probe (e.g., complement). In some embodiments, analysis of the nucleic acid hybridized to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture probe bound to a nucleic acid) from the substrate and/or analyzing a nucleic acid hybridized to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of a nucleic acid hybridized to a capture probe from the substrate.

In some embodiments, provided herein are methods for spatially detecting a nucleic acid (e.g., detecting the location of a nucleic acid) from a biological sample (e.g., present in a biological sample) that include: (a) contacting the biological sample with an array comprising a plurality of capture probes, wherein a capture probe of the plurality captures the nucleic acid; (b) optionally staining and/or imaging a biological sample on a substrate; (c) permeabilizing (e.g., providing a solution comprising a permeabilization reagent to) the biological sample on the substrate and (d) analyzing the captured nucleic acid, thereby spatially detecting the nucleic acid; where the biological sample is not removed from the substrate.

In some embodiments, provided herein are methods for spatially detecting a nucleic acid of interest from a biological sample that include: (a) contacting the biological sample with an array on a substrate, wherein the array comprises one or more capture probe pluralities thereby allowing the one or more pluralities of capture probes to capture the nucleic acid of interest; (b) staining and imaging a biological sample on a substrate: (v) providing a solution comprising a permeabilization reagent to the biological sample on the substrate; and (d) analyzing the captured nucleic acid, thereby spatially detecting the nucleic acid of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APEI), U uracil-specific excision reagent (USER), and/or an endonuclease VIII. In some embodiments, one or more capture probes do not comprise a cleavage domain and is not cleaved from the array.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some instances, the ligated probe and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences: however a skilled artisan will understand that additional or alternative sequences used by other sequencing instruments or technologies are also equally applicable for use in the aforementioned methods.

In some embodiments, where a sample is barcoded directly via hybridization with capture probes or nucleic acids hybridized, bound, or associated with either the cell surface, or introduced into the cell, as described above, sequencing can be performed on the intact sample.

A wide variety of different sequencing methods can be used to analyze barcoded analyte derived products (e.g., the ligation product). In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (1) Kits and Compositions

In some embodiments, also provided herein are kits that include one or more reagents to testing a biological sample for determining the state of the target nucleic acids and their efficient capture and detection, or for determining optimal conditions for detection of target nucleic acids, for use in spatial transcriptomics. In some instances, the kit includes a substrate including a plurality of capture probes including a spatial barcode and the capture domain. In some instances, the kit includes a plurality of probes (e.g., a first probe and a second probe). In some embodiments, the kit includes a substrate including a plurality of capture probes that do not include a spatial barcode and do include a capture domain. In some embodiments, a kit includes a substrate with a plurality of capture probes that include spatial barcodes and capture domains and a second substrate where the plurality of capture probes lack the spatial barcodes.

In some embodiments, the kit includes: (a) a substrate including a plurality of capture probes including a capture domain; (b) a system including: a plurality of first probes and second probes, wherein a first probe and a second probe each includes sequences that are substantially complementary to an rRNA, and wherein the second probe includes a capture binding domain; (c) a plurality of enzymes including a ribonuclease, a ligase, and a polymerase; (d) a plurality of labelled dNTPs; and (e) instructions for performing the method of any one of the preceding methods for determining the state of the nucleic acids in a biological sample, or for determining optimal conditions for detection of target nucleic acids, for use in spatial transcriptomics.

In some embodiments, the kit includes: (a) an array including a plurality of capture probes; (b) a plurality of probes including a first probe and a second probe, wherein the first probe and the second probe are substantially complementary to adjacent sequences of a IRNA, wherein the second probe includes a capture probe capture domain that is capable of hybridizing to a capture domain of the capture probe: an enzyme including (c) a plurality of enzymes including a ribonuclease, a ligase, and a polymerase; (d) a plurality of labelled dNTPs; and (e) instructions for performing the method of any one of the methods for determining the state of the nucleic acids in a biological sample, or for determining optimal conditions for detection of target nucleic acids, for use in spatial transcriptomics.

In some embodiments of any of the kits described herein, the first and second probes include sequences that are substantially complementary to adjacent sequences of an 18S rRNA. In some embodiments of any of the kits described herein, the first probe includes a sequence that is at least 80% identical to SEQ ID NO: 1. In some embodiments of any of the kits described herein, the second probe includes a sequence that is at least 80% identical to SEQ ID NO: 2.

In some embodiments, provided herein are compositions that include one or more reagents to detect one or more nucleic acids as described herein. In some embodiments, a composition includes a spatial array including capture probes, where the capture probes include a capture domain, a biological sample on the spatial array wherein the biological sample includes a plurality of rRNAs of interest, a first probe and a second probe hybridized to the rRNAs of interest and ligated together, where the first probe and the second probe each include a sequence that is substantially complementary to adjacent sequences of the rRNA and where one of the first probe or the second probe includes a capture probe capture domain. In some embodiments, the composition also includes an endoribonuclease. In some embodiments, the composition includes an RNase H enzyme. In some embodiments, the composition further includes an RNase A enzyme. In some embodiments, the composition further includes a ligase. In some embodiments of any of the compositions described herein, one of the first probe or the second probe includes a functional sequence (e.g., any of the exemplary functional sequences described herein). In some embodiments of any of the compositions described herein, the first probe includes a sequence that is at least 80% identical to SEQ ID NO: 1. In some embodiments of any of the compositions described herein, the second probe includes a sequence that is at least 80% identical to SEQ ID NO: 2.

In some embodiments, a composition comprises an array including a plurality of capture probes wherein a capture probe comprises a capture domain, a ligation product hybridized to a capture domain, and a fluorescently labeled extension product hybridized to the ligation product. In some embodiments, the extension product includes one or more functional sequences. In some embodiments, the ligation product includes a sequence that is 80% identical to SEQ ID NO: 1. In some embodiments, the ligation product includes a sequence that is 80& identical to SEQ ID NO:2.

### EXAMPLES

### Example 1. Evaluating FFPE Permeabilization Conditions by mRNA Capture

This example demonstrates that methods used for optimizing permeabilization conditions for a FFPE tissue sample can be measured by capturing the release of mRNA from deparaffinized, decrosslinked, and permeabilized FFPE tissues.

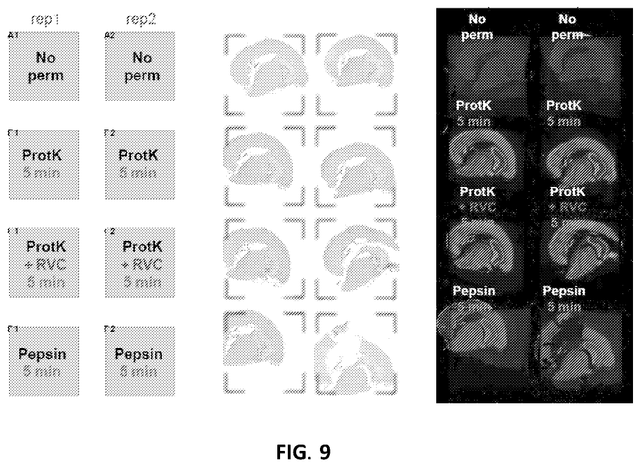

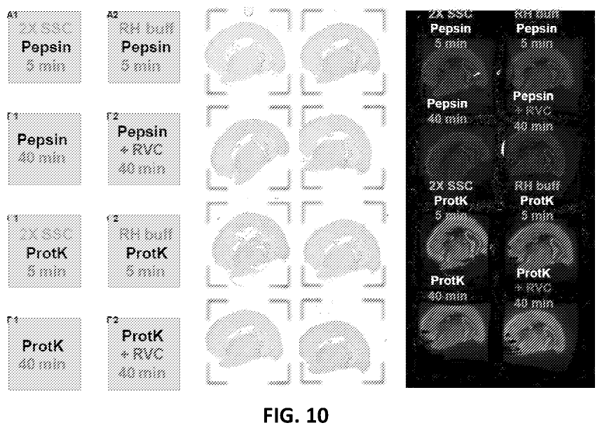

FIGS. 9 and 10 are indicative of using direct mRNA capture for determining efficacy of permeabilization conditions in releasing target nucleic acids from FFPE samples.

Using mouse brain tissue sections, permeabilization conditions were varied and analyte capture determined. Variables evaluated included: 1) pepsin as the permeabilization agent, proteinase K as the permeabilization agent, or no permeabilization agent, 2) time of permeabilization was varied, using either 5 min or 40 min, 3) addition of ribonuclease inhibitor RVC (ribonucleoside vanadyl complexes), and 4) SSC solution or RNase H (RH) buffer as the general tissue buffer prior to tissue permeabilization and use of permeabilization agents in their respective buffers.

Briefly, tissue sections of FFPE mouse brain were placed on a slide containing capture probes with capture domains. The tissue sections were deparaffinized by incubating the slides for 30 min at 60C followed by a series of xylene and ethanol washes. Following deparaffinization, the tissues were H&E stained and imaged.

Post imaging, the tissues were decrosslinked. Briefly, tissues were washed with a 0. IN HCl wash and decrosslinked at 70C for around 60 min in a solution of TE, pH 8.0, followed by PBS washes. Following decrosslinking of the tissues, the tissues were permeabilized following the experimental parameters found in FIGS. 9 and 10 (left panels). Pepsin or proteinase K (proK) enzymes in their respective buffers were used as the permeabilization reagents, with or without RVC present in the permeabilization reagent, with permeabilization times being either 5 min or 40 min at 37C. Briefly, decrosslinked tissues in either SSC or RH buffers were washed with PBS wash solution just prior to addition of the respective permeabilization reagents. The permeabilization reagents were added to the tissues follow by either 5 min or 40 min additional incubation. The permeabilization reagents were added to tissues such that, regardless of incubation time, permeabilization ended simultaneously. The tissues were washed with SSC. Control tissue sections (FIG. 9 “No perm”) went through all the experimental steps except the permeabilization steps.

Following the different permeabilization conditions, mRNA capture was allowed to proceed, the captured mRNA were extended, tissue removed, and fluorescence imaging detected as described in the Visium Spatial Gene Expression Reagent Kits-Tissue Optimization user guide (10×Genomics).

FIGS. 9 and 10, center and right, show the tissues imaged following H&E staining and fluorescence imaging, respectively, which align with the experimental parameters, left. As seen in FIG. 9, following deparaffinization all the tissue section regardless of experimental condition stained comparably with H&E. The same was seen in the H&E stained tissues in FIG. 10. FIG. 9 served as a control slide. The negative controls (No perm) showed some background RNA capture and extension, however the 5 min incubations using pepsin or proK, with or without RVC, were all comparable in fluorescent signal. The proteinase K permeabilization was perhaps blurrier but with more intense signal that pepsin, whereas the pepsin permeabilization yielded crisper images. FIG. 9 demonstrates that time of permeabilization is an important factor, as is buffer choice depending on the permeabilization enzyme, when compared to FIG. 10. It was determined that 5 min permeabilization time was sufficient for mouse brain and that the mRNA remains intact, resulting in bright fluorescent images. However, it is contemplated that prolonged incubation time, especially noticeable when pepsin is used to permeabilize, could be degrading the mRNA (whether enzyme is present or not), resulting in images with diminished fluorescence. Additionally, time of permeabilization for pepsin also affected mRNA capture. For example, compared to a 5 min permeabilization time for pepsin, the 40 min permeabilization time shows almost no signal. The longer permeabilization time when using proteinase K resulted in blurrier images. It is thought that the longer permeabilization time may be causing RNA degradation. RVC may decrease the degradation as seen in FIG. 10 where signal is higher and crisper at 40 min with proteinase K when RVC is present, whereas the same experiments done for 5 min did not appear to substantially affect the fluorescent signal.

As such, FIGS. 9 and 10 demonstrate that it is possible to use mRNA capture as a method for evaluating permeabilization conditions for FFPE fixed samples, as well as its use as a method for evaluating conditions which guard against RNA degradation.

### Example 2. Evaluating FFPE Permeabilization Conditions by RNA Templated Ligation

This example demonstrates that methods used for optimizing permeabilization conditions for a FFPE tissue sample can be measured by practicing RNA templated ligation methods on deparaffinized, decrosslinked and permeabilized FFPE tissues. The method described in this example increased the predictability for RTL gene expression results based on permeabilization condition optimization.

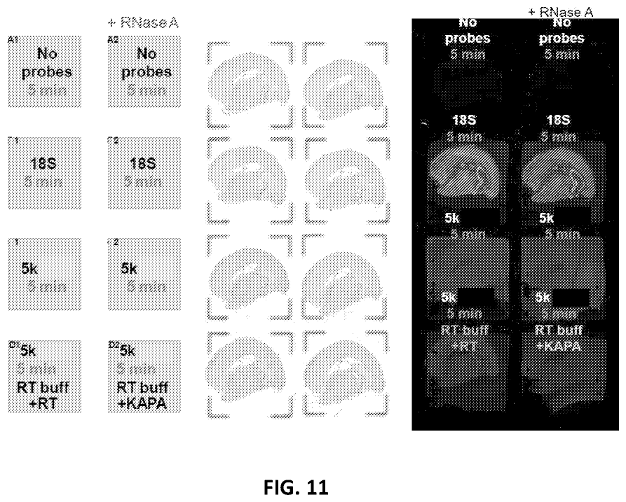

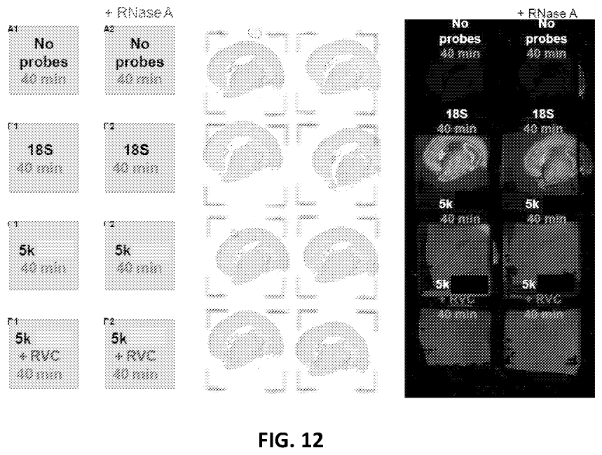

FIGS. 11-12 are indicative of using an RTL approach for determining efficacy of permeabilization conditions on release of target nucleic acids from FFPE tissues.

Mouse brain tissue sections were used in these experiments. The variables included permeabilization time (5 min versus 40 min), probe type (RTL probes to 5000 RNA targets versus 18S probes), presence or absence of RVC, and enzyme used for second strand synthesis (reverse transcriptase versus Kapa HiFi DNA polymerase).

Briefly, tissue sections from FFPE mouse brain were exposed to the same deparaffinization, H&E staining and imaging as in Example 1. The tissues were decrosslinked as in Example 1, and probes, either RTL probes to 5000 mRNA targets (approximately 1.2 nM of each probe, therefore approximately 5 mM total probes) or an 18S probe pair (2 mM each probe), were added to the decrosslinked tissue sections in a SSC hybridization buffer, tissues were incubated at 50° C. for about 2½ hours, and washed. The hybridized probes were ligated by adding to the tissues a ligation mix including a ligase and buffer, incubating at 37° C. for around 1 hour followed by washing the tissues in SSC wash solution. Following RTL probe ligation, the hybridized and ligated ligation products were released from their targets and the tissues were permeabilized. Briefly. RNase H was added to the tissues and incubated at 37 C for around 30 min, after which proteinase K was added and incubation was continued for another 5 min. The tissues were washed with SSC buffer and extension of the captured ligation product, or second strand synthesis, was performed.

Two second strand synthesis buffers. Second Strand-A (SS-A) and Second Strand-B (SS-B), were evaluated for their ability to create a labeled complement of the captured ligation product. Both buffers included fluorescently labeled dCTP. Second strand-A buffer further included KAPA HiFi DNA polymerase in a buffer and is optimized more for a DNA thermal polymerase than is the SS-B buffer. SS-B further included Reagent C used in the Visium Reverse Transcriptase reaction (Visium Spatial Gene Expression Reagent Kits-Tissue Optimization user guide (10×Genomics) and KAPA HiFi DNA polymerase. Reverse transcriptase was also evaluated for second strand synthesis, using reagents from the Visium Spatial Gene Expression Reagent Kits-Tissue Optimization kit. The appropriate second strand synthesis reaction mix was added to the tissues and the extension reactions were performed at 53° C. for around 25 min followed by washing of the tissue samples. Tissues were removed and the extension products were fluorescently imaged.

FIG. 11 mainly serves as a control slide where permeabilization occurred for 5 min. As seen in FIG. 11, the no probe controls had no background. The 18S probes showed successful incorporation of the labeled nucleotides resulting in crisp images. However, the 5000 (5 k) probes target experiments regardless of enzyme or buffer did not perform as well as the 18S probes and did not provide useful information. This is duplicated in FIG. 12. As only a few percent of total RNA is mRNA, it is contemplated that the non-18S probe targets were lower in abundance than the 18S target. Further, in contrast to typical mRNA length the 5000 RTL ligation products were much shorter and may not have incorporated labelled nucleotides. As such, these results demonstrate that using more abundant targets, such as 18S, is preferential for use in the methods of determining which permeabilization conditions would correlate to acceptable downstream spatial gene expression profiles.

FIG. 12 shows results for a 40 min permeabilization time. For the 40 min experiments, the RNase inhibitor RVC was added to one set of 5 k probe tissues, however it did not appear to have any effect on fluorescence outcome. As such, it is contemplated that the results using the 5 k RTL probes are probably not due to RNA degradation. On the other hand, the 40 min incubation in combination with the 18S probes still provides for good fluorescent signal.

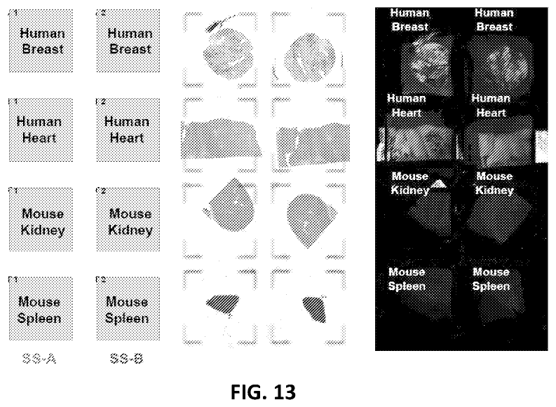

FIG. 13 shows results when the RTL methods were applied to alternative tissue types to mouse brain, these experiments include evaluating the SS-A and SS-B extension reaction reagents. Both human breast and human heart showed successful RTL ligation product capture and extension, with slightly better fluorescent signal when using SS-A compared to SS-B. Mouse kidney and mouse spleen experiments appear to have resulted in minimal RTL ligation product capture and extension products, seen as minimal to no fluorescent signal (e.g., below the level of detection, which could potentially be increased by increasing exposure time).

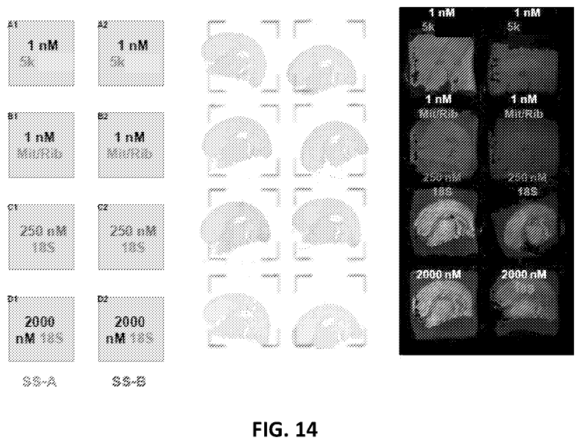

FIG. 14 shows results of mouse brain tissues using RTL methods with different concentrations of RTL probes: 5 k probes (control), mitochondrial/ribosomal targeted probes, or 18S probes. The left four array areas used SS-A extension reagent, whereas the right four array areas used SS-B extension reagents. The 18S probes provided enhanced target capture and extension of the 18S RTL ligation products compared to the capture and extension of the 5 k RTL ligation products and the mitochondrial/ribosomal RTL ligation products. In this experiment, while both conditions yielded fluorescence signal and were therefore successful, the SS-A conditions for extension were optimal to the SS-B extension reagents, regardless of probe set, yielding crisper and slightly brighter images.

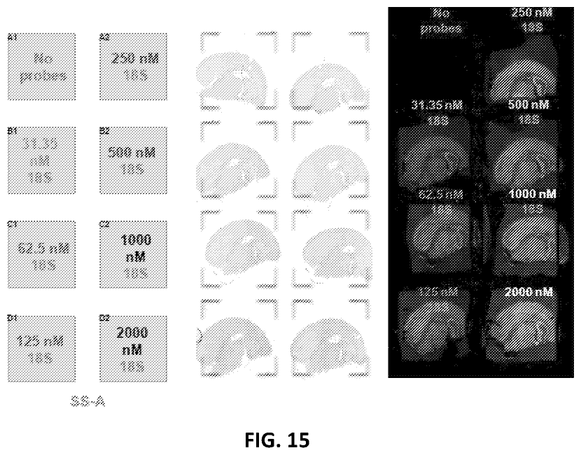

FIG. 15 shows results of mouse brain tissues using RTL methods with different concentrations of 18S probes (or no probes as the negative control), in combination with SS-A extension reagent. As seen in the right hand panels, the negative control did not show any fluorescent signal. In this experiment, as the concentration of the 18S probes increased so did the fluorescent signal, demonstrating an increase in capture and extension of the 18S RTL ligation products in combination with the SS-A buffer.

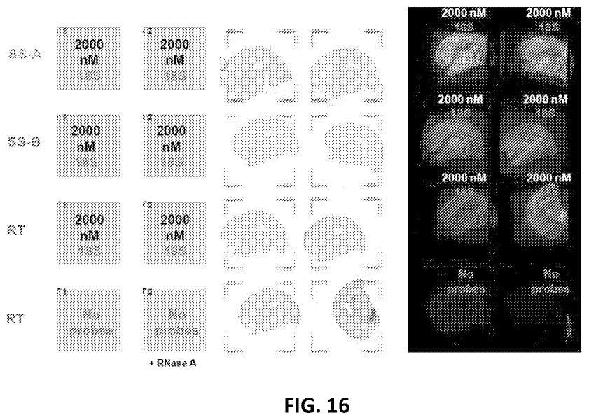

FIG. 16 shows results of mouse brain tissues using RTL methods comparing the three different buffers for extending captured products (e.g., SS-A with KAPA HiFi DNA polymerase, SS-B with KAPA HiFi DNA polymerase, and RT with reverse transcriptase), with and without RNase A that was supplemented into the RNase H buffer. In this experiment, the 18S probe concentration was kept constant at 2 mM. The need for RNase A was also evaluated as single stranded RNA might be present and bind the capture probes on the array in addition to the RTL ligation products, thereby creating non-specific background binding. The experiment also including no probe negative controls. In some experiments that lacked the RNase A, there was very little background fluorescence seen even with the no probe control. It is contemplated that the low background signal is potentially due to a small amount of free mRNA that was bound to the surface capture probes and extended. On the other hand, when RNase A was applied (bottom right image of FIG. 16), no background fluorescence was seen. As previously demonstrated, capture and extension of the ligated RTL 18S probes was increased when using the SS-A buffer and KAPA HiFi DNA polymerase, compared to the SS-B with the KAPA enzyme or the reverse transcriptase enzyme in (RT) buffer Visium RT reagent C. The KAPA HiFi DNA polymerase is not known to synthesize from the RNA strand and the controls do not show background fluorescence when that enzyme is used. As such, when working with SS-A buffer and the KAPA HiFi DNA polymerase. RNase A is not needed. KAPA HiFi DNA polymerase in combination with the SS-A buffer was determined to be the superior method in these experiments. The addition of RNase A has an impact on decreasing background when the reverse transcriptase in RT buffer is used. Further, while the RT experiments yielded usable results, the images are not as crisp comparative to when KAPA HiFi DNA polymerase is used.

### Example 3. Evaluating FFPE Permeabilization Conditions by RNA Templated Ligation Followed by Gene Expression Analysis

After determining that the methods disclosed herein could be used to identify successful permeabilization conditions in FFPE tissues and conditions that were preferred in preserving RNA integrity, experiments were undertaken to evaluate downstream gene expression analysis following those methods.

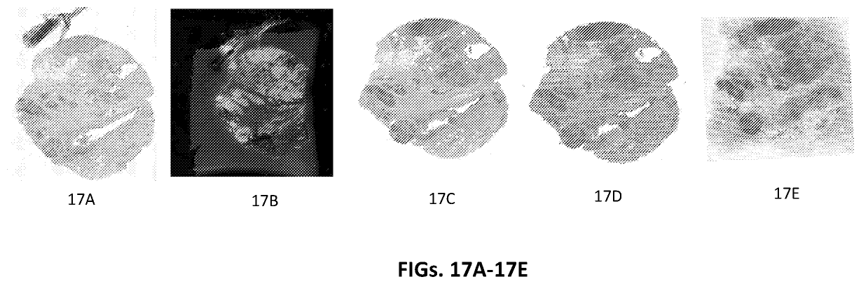

Briefly, tissue sections from human breast cancer tissue samples were prepared and stained as found in Example 2 using 2 mM 18S probes and KAPA HiFi DNA polymerase in SS-A buffer to determine permeabilization conditions. FIG. 17A shows H&E stained human breast cancer tissue section and FIG. 17B the resultant permeabilization followed by fluorescence of the capture and extension of the 18S RTL ligation products.

FIGS. 17C-17E show the results of the gene expression analysis. Briefly, a second human breast cancer tissue section from the same sample block that was used for the permeabilization experiments was deparaffinized, stained and decrosslinked as previously described. A whole transcriptome set of RTL probes in a hybridization buffer were added to the decrosslinked tissue section and hybridization was allowed to proceed overnight, followed by ligation of the RTL probes at 37 C for 1 hr. The tissue sample was washed and exposed to permeabilization conditions, as previously defined, and the RTL ligation products were released from the target RNA sequences and allowed to hybridize to the capture domains on the capture probes affixed to the spatial array slides. Following capture, the RTL ligation products were extended using KAPA HiFi DNA polymerase in SS-A buffer without labeled nucleotides. The tissue was removed from the slide and gene expression analysis was determined. FIG. 17C shows the H&E stained tissue, FIG. 17D represents the clustered gene expression analysis and FIG. 17E the gene expression heat map of UMI counts (log 10). This experiment demonstrates that the methods described herein for determining permeabilization conditions can be successfully applied for use in an RTL workflow as well as a direct mRNA capture workflow.

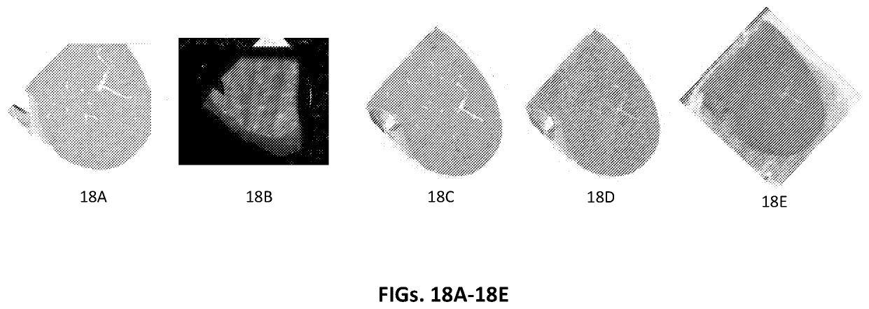

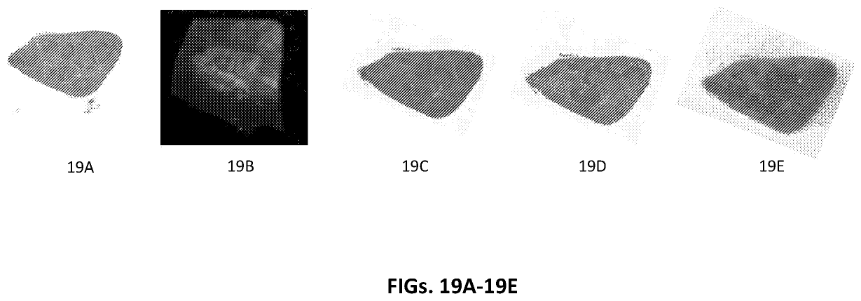

FIGS. 18A-18E and FIGS. 19A-19E show gene expression analysis results for mouse kidney and spleen, respectively. Briefly, for the fluorescent assay for both kidney and spleen samples, 18S (2 mM) probes were used with KAPA HiFi DNA polymerase in SS-A as described in Example 2 and as utilized for the breast cancer samples. For expression analysis for the kidney samples, a whole transcriptome set of RTL probes was used whereas for the spleen samples a reduced set of 3000 probes was used. The samples were processed as above, regardless of sample type.

While the 18S RNA, and perhaps rRNA in general, may have different properties than the mRNAs in localization and abundance, the results demonstrate that the methodology described herein still serves as a good approximation for mRNA expression analysis. The fluorescent footprints seen from the fluorescent RTL based assays correlate with expression analysis UMI counts as seen in the heat maps (FIGS. 17E, 18E, and 19E) from gene expression analysis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining a reaction condition for efficiently detecting a target nucleic acid in a biological sample, the method comprising:
(a) contacting the biological sample on an array comprising a plurality of capture probes, wherein a capture probe of the plurality comprises a capture domain;
(b) contacting a first probe and a second probe with the biological sample, wherein the first probe and the second probe each comprise sequences that are substantially complementary to sequences of the target nucleic acid, and wherein the second probe comprises a capture probe capture domain;
(c) hybridizing the first probe and the second probe to the target nucleic acid;
(d) generating a ligation product by ligating the first probe and the second probe;
(e) hybridizing the ligation product to the capture domain;
(f) generating a sequence that is complementary to the hybridized ligation product wherein the complementary sequence comprises one or more labeled nucleotides;
(g) detecting intensity of a signal; and
(h) correlating the intensity of the signal with the efficiency of incorporation of the one or more labelled nucleotides into the generated sequence thereby determining the reaction condition for efficiently detecting the target nucleic acid in the biological sample.

2. A method for identifying permeabilization conditions for a biological sample on an array, the method comprising:
(a) contacting the biological sample on the array, wherein the array comprises a plurality of capture probes, wherein a capture probe of the plurality comprises a capture domain;
(b) contacting a first probe and a second probe with the biological sample, wherein the first probe and the second probe each comprise sequences that are substantially complementary to sequences of a target nucleic acid, and wherein the second probe comprises a capture probe capture domain;
(c) hybridizing the first probe and the second probe to the target nucleic acid;
(d) generating a ligation product by ligating the first probe and the second probe; (e) permeabilizing the biological sample;
(f) hybridizing the ligation product to the capture domain;
(g) generating a sequence that is complementary to the hybridized ligation product wherein the complementary sequence comprises one or more labeled nucleotides; and
(h) correlating the intensity of a signal associated with the labelled nucleotides in the generated sequence with the permeabilization condition, thereby identifying permeabilization conditions for the biological sample.

3. The method of claim 1 or 2, wherein the correlating the intensity of the signal further determines degradation status of the target nucleic acid.

4. The method of any one of claims 1-3, wherein the generating step comprises extending the capture probe using the ligation product as a template, thereby generating a sequence that is complementary to the ligation product.

5. The method of any one of claims 1-4, wherein the first probe and the second probe are substantially complementary to adjacent sequences of the target nucleic acid.

6. The method of any one of claims 1-4, where the first probe and the second probe hybridize to sequences that are not adjacent to each other on the target nucleic acid.

7. The method of claim 6, wherein the first probe is extended with a DNA polymerase, thereby (i) filling in a gap between the first probe and the second probe and (ii) generating an extended first probe.

8. The method of any one of the preceding claims, wherein the generating step comprises contacting the ligation product with one or more of:
an enzyme selected from a DNA polymerase, a phi29 DNA polymerase, a thermostable DNA polymerase or a reverse transcriptase;
a buffer; and
a plurality of nucleotides (dNTPs), wherein the plurality of nucleotides comprises one or more labelled nucleotides.

9. The method of claim 8, wherein the reverse transcriptase is selected from the group consisting of: avian myeloblastosis virus (AMV) reverse transcriptase, Moloney murine leukemia virus (M-MuLV or MMLV) reverse transcriptase, and HIV reverse transcriptase, or functional variants thereof.

10. The method of claim 9, wherein the reverse transcriptase is an M-MLV reverse transcriptase enzyme.

11. The method of any one of the preceding claims, wherein the plurality of nucleotides comprise one or more of a dATP, a dTTP, a dGTP, a dUTP, and a dCTP.

12. The method of any one of the preceding claims, wherein the labelled nucleotides comprise one or more of a labelled dATP, a labelled dTTP, a labeled dUTP, a labelled dGTP, or a labelled dCTP.

13. The method of any one of the preceding claims, wherein labelled nucleotides comprise a label selected from the group consisting of: a radiolabel, a fluorescent label, a chemiluminescent label, a bioluminescent label, a calorimetric label, and a colorimetric label.

14. The method of claim 16, wherein the fluorescent label comprises a fluorophore selected from the group consisting of: Cy3, Cy5, Cy5.5, Cy7, TAMRA, 5-ROX, TYE 653, HEX, TEX 615, TYE 665, and TYE 705.

15. The method of any one of the preceding claims, wherein the target nucleic acid comprises RNA.

16. The method of claim 15, wherein the level of the RNA is transcribed from a housekeeping gene.

17. The method of claim 15 or 16, wherein the RNA is a ribosomal RNA (rRNA).

18. The method of claim 17, wherein the rRNA is selected from the group consisting of 18S, 28S, 5.8S, 5S, 12S, 16S, and 23S.

19. The method of claim 17 or 18, wherein the rRNA is 18S.

20. The method of any one of claims 17-19, wherein the first probe comprises a sequence that is substantially complementary to a first sequence of the IRNA.

21. The method of any one of claims 17-20, wherein the second probe comprises a sequence that is substantially complementary to a second sequence of the rRNA.

22. The method of any one of the preceding claims, wherein the first probe comprises a sequence that is at least 80% identical to SEQ ID NO: 1.

23. The method of claim 22, wherein the first probe comprises a sequence of SEQ ID NO: 1.

24. The method of any one of the preceding claims, wherein the second probe comprises a sequence that is at least 80% identical to SEQ ID NO: 2.

25. The method of claim 24, wherein the second probe comprises a sequence of SEQ ID NO: 2.

26. The method of any one of the preceding claims, wherein the first probe further comprises a functional sequence, wherein the functional sequence is a primer sequence.

27. The method of any one of the preceding claims, wherein the biological sample is contacted with the first probe and the second probe at a total concentration of about 25 nM to about 2500 nM.

28. The method of claim 27, wherein the biological sample is contacted with the first probe and the second probe at a total concentration of about 100 nM to about 2000 nM.

29. The method of any one of the preceding claims, wherein the first probe and/or the second probe is a DNA probe.

30. The method of any one of the preceding claims, further comprising contacting the biological sample with one or more additional probe pairs, wherein a probe pair comprises an additional first probe and an additional second probe.

31. The method of claim 30, wherein each of the one or more additional probe pairs comprises (i) a first sequence that is substantially complementary to a sequence of the IRNA, and (ii) a second sequence that is substantially complementary to a sequence of the rRNA, wherein the first sequence and the second sequence are adjacent in the rRNA.

32. The method of claim 30, wherein each of the one or more additional probe pairs comprises (i) a first sequence that is substantially complementary to a sequence of a second rRNA, and (ii) a second sequence that is substantially complementary to a sequence of a second rRNA, wherein the first sequence and the second sequence are adjacent in the second rRNA.

33. The method of claim 30, wherein each of the one or more additional probe pairs comprises (i) a first sequence that is substantially complementary to a sequence of a second target nucleic acid, and (ii) a second sequence that is substantially complementary to a sequence of the second target nucleic acid.

34. The method of any one of the preceding claims, wherein generating a ligation product comprises ligating the first probe to the second probe, wherein the enzymatic ligation utilizes a ligase.

35. The method of claim 34, wherein the ligase is one or more of a T4 RNA ligase (Rnl2), a PBCV-1 ligase, a ligase from a Chlorella virus, a single stranded DNA ligase, or a T4 DNA ligase.

36. The method of any one of claim 1 or 3-35, wherein the method further comprises permeabilizing the biological sample.

37. The method of claim 2 or 36, wherein permeabilizing comprises contacting the biological sample with a permeabilization agent.

38. The method of claim 37, wherein the permeabilization agent is selected from an organic solvent, a detergent, and an enzyme, or a combination thereof.

39. The method of claim 37, wherein the permeabilization agent is selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, a nonionic surfactant, and polyoxyethylene sorbitol.

40. The method of claim 37, wherein permeabilizing agent comprises an endopeptidase.

41. The method of claim 40, wherein the endopeptidase is pepsin or proteinase K.

42. The method of any one of the preceding claims, further comprising providing a capture probe capture domain blocking moiety that interacts with the capture probe capture domain.

43. The method of claim 42, further comprising releasing the capture probe capture domain blocking moiety from the capture probe capture domain prior to the step of hybridizing the ligation product to the capture domain.

44. The method of any one of the preceding claims, wherein the capture probe capture domain comprises a poly-adenylated (poly(A)) sequence or a complement thereof.

45. The method of claim 44, wherein the capture probe capture domain blocking moiety comprises a poly-uridine sequence, a poly-thymidine sequence, or both.

46. The method of claim 45, wherein releasing the poly-uridine sequence or poly-thymidine sequence from the poly(A) sequence comprises denaturing the ligation product or contacting the ligation product with an endonuclease, exonuclease or ribonuclease.

47. The method of any one of the preceding claims, wherein the capture probe capture domain comprises a sequence that is complementary to all or a portion of the capture domain of the capture probe.

48. The method of any one of the preceding claims, wherein the capture probe capture domain blocking moiety is a DNA probe.

49. The method of any one of the preceding claims, further comprising removing the biological sample from the substrate.

50. The method of claim 49, wherein the removing step is performed prior to the step of hybridizing the ligation product to the capture domain.

51. The method of claim 49, wherein the removing step is performed prior to the step of generating a sequence that is complementary to the hybridized ligation product.

52. The method of any one of the preceding claims, wherein the signal corresponding to the bound ligation product comprises the signal from the labeled dNTPs.

53. The method of any one of the preceding claims, wherein the detecting step comprises obtaining an image corresponding to the signal corresponding to the bound ligation product on the substrate.

54. The method of claim 53, further comprising registering image coordinates to a fiducial marker.

55. The method of any one of the preceding claims, further comprising analyzing a signal corresponding to the bound ligation product on the substrate.

56. The method of any one of the preceding claims, further comprising identifying, based on the signal analysis, the quality of the biological sample.

57. The method of any one of the preceding claims, wherein the biological sample is a tissue sample.

58. The method of claim 57, wherein the tissue sample is a formalin-fixed, paraffin-embedded (FFPE) tissue sample.

59. The method of claim 57 or 58, wherein the tissue sample is the FFPE tissue sample, and the tissue sample is decrosslinked.

60. The method of any one of the preceding claims, wherein the biological sample was previously stained.

61. The method of claim 60, wherein the biological sample was previously stained using immunofluorescence or immunohistochemistry.

62. The method of claim 61, wherein the biological sample was previously stained using hematoxylin and eosin.

63. The method of any one of the preceding claims, further comprising selecting, based on the detected signal, a second tissue section of the biological sample for the detection of one or more additional target nucleic acids.

64. The method of claim 63, wherein the second tissue section is a serial section of the biological sample.

65. The method of any one of the preceding claims, further comprising analyzing the second portion of the biological sample for the detection of one or more additional target nucleic acids, wherein analyzing the second portion comprises determining a location and abundance of one or more target nucleic acids in the second portion of the biological sample.

66. The method of claim 65, wherein the steps of analyzing comprise:
(a) providing the second tissue section of the biological sample on a second array comprising a second plurality of second capture probes, wherein a second capture probe of the second plurality of second capture probes comprises: (i) a spatial barcode and (ii) a second capture domain;
(b) hybridizing a second plurality of probes with the biological sample, wherein a first probe of the second plurality of probes and a second probe of the second plurality of probes each comprise sequences that are substantially complementary to sequences of the target nucleic acid, and wherein the second probe of the second plurality of probes comprises a second capture domain;
(c) generating a second ligation product by ligating the first probe of the second plurality of probes and the second probe of the second plurality of probes;
(d) hybridizing the second ligation product to the second capture domain; and
(e) determining (i) all or a part of the sequence of the second ligation product bound to the capture domain, or a complement thereof, and (ii) the spatial barcode, or a complement thereof, and using the determined sequence of (i) and (ii) to identify the location of the target nucleic acid in the biological sample.

67. The method of any one of the preceding claims, further comprising a releasing step that comprises removing the ligation product from the nucleic acid prior to hybridizing the ligation product to the capture domain.

68. The method of claim 67, wherein the releasing of (i) the ligation product from the target nucleic acid or (ii) the capture probe capture domain blocking moiety from the capture domain binding domain, comprises contacting the ligated probe with an endoribonuclease.

69. The method of claim 68, wherein the endoribonuclease is one or more of RNase H, RNase A, RNase C, or RNase I.

70. The method of claim 68 or 69, wherein the endoribonuclease is RNase H.

71. The method of claim 69 or 70, wherein the RNase H comprises RNase H1, RNase H2, or RNase H1 and RNase H2.

72. The method of any one of claim 2 or 36-71, wherein the permeabilizing the biological sample occurs before contacting a first probe and a second probe with the biological sample.

73. The method of any one of claim 2 or 36-72, wherein the permeabilizing the biological sample occurs before releasing the ligation product from the target nucleic acid.

74. The method of any one of claim 2 or 36-73, wherein permeabilizing the biological sample occurs after releasing the ligation product from the target nucleic acid.

75. A kit comprising:
(a) a substrate comprising a plurality of capture probes comprising a spatial barcode and a capture domain;
(b) a plurality of first probes and second probes, wherein a first probe and a second probe each comprises sequences that are substantially complementary to an rRNA, and wherein the second probe comprises a capture binding domain;
(c) a plurality of enzymes comprising a ribonuclease, a ligase, and a polymerase
(d) a plurality of labelled dNTPS; and
(e) instructions for performing the method of any one of the preceding claims.

76. A kit comprising:
(a) an array comprising a plurality of capture probes;
(b) a plurality of probes comprising a first probe and a second probe, wherein the first probe and the second probe are substantially complementary to adjacent sequences of a rRNA, wherein the second probe comprises a capture probe capture domain that is capable of binding to a capture domain of the capture probe;
an enzyme comprising
(c) a plurality of enzymes comprising a ribonuclease, a ligase, and a polymerase;
(d) a plurality of labelled dNTPs; and
(e) instructions for performing the method of any one of the preceding claims.

77. The kit of claim 75 or 76, wherein the rRNA is 18S.

78. The kit of any one of claims 75-77, wherein the first probe comprises a sequence that is at least 80% identical to SEQ ID NO: 1.

79. The kit of any one of claims 75-78, wherein the second probe comprises a sequence that is at least 80% identical to SEQ ID NO: 2.
